(19) United States
(12) Patent Application Publication
(10) Pub. No.: US 2007/0078161 A1
(43) Pub. Date: Apr. 5, 2007

(54) Crystalline forms of (+)- and (-) erythro-mefloquine hydrochloride

(76) Inventors: Kenneth Walter Sinden, Essex (GB); Andrew Douglas Baxter, Essex (GB); Martin Szelagiewicz, Bassel (CH); Rolf Hilfiker, Basel (CH)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/575,998

(22) PCT Filed: Dec. 17, 2004
(86) PCT No.: PCT/GB04/05331

§ 371 Date: Oct. 16, 2006

(30) Foreign Application Priority Data
Dec. 17, 2003 (GB) 0329236.4

(51) Int. Cl. A61K 31/4709; C07D 401/02

(52) U.S. Cl. 514/314; 546/176

(57) ABSTRACT

(+)- or (−)-erythro-Mefloquine hydrochloride can exist in four crystalline forms A, B, C and D, whereby form A is the most stable form. Form A can be directly produced in morphological forms like thick columns, cuboids, cubes and cube-like forms, which can be easily handled during processing and formulation. (+)- or (−)-eroryth-Mefloquine hydrochloride also forms solvates with acetone, methyl ethyl ketone and tetrahydrofuran.

### FIELD OF THE INVENTION

The present invention relates to stable crystalline forms of (+)- and (−)-erythro-mefloquine hydrochloride, preferably in an easy to handle morphology.

### BACKGROUND TO THE INVENTION

(+)- and (−)-erythro-Mefloquine are the trivial names for (+)-(11S,2′R)α-2-piperidinyl-2,8-bis(trifluoromethyl)-4-quinolinemethanol (2) and (−)-11R,2′S)α-2-piperidinyl-2,8-bis(trifluoromethyl)-4quinolinemethanol (1) of formulae

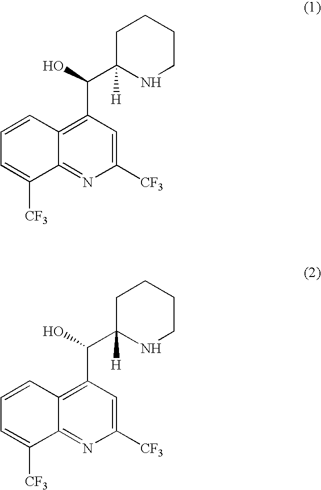

Mefloquine is a chiral drug substance and synthetic analogue of quinine, originally developed to replace existing anti-malarials where resistance had developed. Although mefloquine is marketed as a racemic mixture, both enantiomers of the drug have been shown to demonstrate different biological activities. EP-A-0966285 discloses (+)-mefloquine for the treatment of malaria with reduced side-effects, while EP-A-0975345 and EP-A-1 107761 disclose that (−)-mefloquine may block purinergic receptors and have utility in the treatment of movement and neurodegenerative disorders. More recently, W002/19994 discloses that (+)-(11S,2′R)-erythro-mefloquine (2) is the preferred enantiomer for the treatment of inflammatory and autoimmune diseases such as rheumatoid arthritis, osteoarthritis, psoriatic arthritis, psoriasis, Crohn's disease, systemic lupus erythematosis (SLE), ulcerative colitis, chronic obstructive pulmonary disease (COPD) and asthma.

J. M. Karle et al, Antimicrobial Agents and Chemotherapy Vol. 46 (5), pages 1529 to 1534 (2002), describes the preparation of (−)-mefloquine hydrochloride hydrate in the form of clear rectangular needles by crystallisation of (−)-mefloquine hydrochloride from a mixture of ethanol and water acidified to pH 2.3 with HCl, and the product's X-ray crystallographic characterisation. Our own investigations have shown that, in contrast to the behaviour of the racemate, the pure enantiomers do not form hydrates. The described form could not be reproduced. The calculated diffraction pattern from the reported single crystal data reveal that it is quite different from the novel form A reported below.

F. 1. Carroll et al, in Journal of Medicinal Chemistry Vol. 17(2), pages 210 to 219, describes the conversion of the free bases of (+)- and (−)-mefloquine with methanolic HCl to the hydrochloride salts of (+)- and (−)-mefloquine and the subsequent re-crystallisation from a CH₂Cl₂ and CH₃CN mixture. The isolated solid product is dried at 100° C., yielding an unstable crystalline compound. It has been found that it is a mixture of crystalline forms described below as B and C (very fine particles).

### SUMMARY OF THE INVENTION

Results obtained during development of (+)-mefloquine hydrochloride indicated that the crystalline compound can be prepared in polymorphic and pseudo-polymorphic forms. It was further surprisingly found that a stable crystalline form, hereinafter called crystalline form A, can be prepared under controlled crystallization conditions and that form A can be prepared by a reliable method in a morphological form which is easy to handle and to process in the manufacture and preparation of pharmaceutical formulations.

Aspects of the present invention include a stable crystalline form A of (+)-and (−)-mefloquine hydrochloride and processes for the preparation thereof in an easy to handle morphology. The use of controlled crystallization conditions allows for an improved production cycle for (+)- and (−)-mefloquine hydrochloride (which, for the purposes of this specification, is understood to be (+) or (−)-erythro-mefloquine hydrochloride).

Crystalline form A of (+)- or (−)-mefloquine hydrochloride comprises a melting point of about 284° C. under decomposition, measured by Differential Scanning Calorimetry with a heating rate of 10° C./minute. The melting point is about 7° C. higher than reported by Carroll et al, supra which is however not a sufficient differentiation due to the fast decomposition. This form A is the most stable form, compared to forms B and C, which is shown suspension experiments with mixtures of forms A, B and C in a temperature range of 2° C. to 75° C. Crystal form C is the least stable form.

Form A

Form A is a crystalline form of (+)- or (−)-mefloquine hydrochloride which exhibits a characteristic X-ray powder diffraction pattern with characteristic peaks expressed in d-values (Å), measured with Synchrotron X-ray radiation:
5.95 (s) and 4.02 (w).

In a further embodiment, Form A is a crystalline form of (+)- or (−)-mefloquine hydrochloride, which exhibits a characteristic Synchrotron X-ray powder diffraction pattern with characteristic peaks expressed in d-values (Å): 11.2 (vs), 9.0 (s), 7.4 (w), 6.8 (w), 6.3 (s), 6.1 (m), 6.0 (m), 5.95 (s), 5.58 (m), 5.42 (m), 4.91 (m), 4.87 (w), 4.74 (s), 4.55 (w), 4.16 (vs), 4.12 (s), 4.10 (s), 4.02 (w), 3.82 (vs), 3.77 (w), 3.74 (s), 3.71 (vs), 3.64 (m), 3.47 (w), 3.40 (w), 3.33 (w), 3.31 (m), 3.27 (w), 3.25 (w), 3.11 (m), 3.04 (m), 2.94 (m), 2.92 (w), 2.75 (w), 2.70 (m), 2.68 (w), 2.64 (m), 2.62 (m), 2.54 (w), 2.45 (w), 2.39 (w), 2.35 (w), 2.30 (w), 2.29 (w), 2.25 (w), 2.22 (w), 2.18 (w), 2.17 (w), 2.08 (w), 1.99 (m), 1.95 (w), 1.91 (w), and 1.88 (w).

In another embodiment, Form A is a crystalline form of (+)- or (−)-mefloquine hydrochloride which exhibits a characteristic X-ray powder diffraction pattern with characteristic peaks expressed in d-values (Å), when using large-sized particles of a size distribution of 30 to 150 μm:
22.3 (vw), 11.2 (vs), 9.0 (w); 8.2 (vw), 7.4 (vw), 6.8 (vw), 6.5 (vw), 6.3 (vw), 6.1 (vw), 6.0 (vw), 5.94 (vw), 5.61 (m), 5.42 (w), 4.89 (vw), 4.74 (w), 4.54 (w), 4.12 (s), 4.02 (w), 3.81 (vvs), 3.74 (vs), 3.70 (vw), 3.64 (vw), 3.55 (w), 3.47 (vw), 3.40 (vw), 3.34 (vw), 3.31 (vw), 3.26 (vs), 3.11 (vw), 3.04 (w), 2.97 (vw), 2.94 (vw), 2.81 (vw), 2.75 (m), 2.71 (w), 2.69 (w), 2.64 (w), 2.62 (w), 2.54 (vw), 2.46 (vw), 2.43 (vw), 2.40 (vw), 2.35 (vw), 2.30 (vw), 2.27 (vw), 2.24 (vw), 2.22 (vw), 2.17 (vs), 2.08 (vw), 2.06 (vw), 2.04 (vw), 1.94 (w), 1.91 (vw) and 1.88 (vw).

Here and in the following the abbreviations in brackets mean: (ws)=very very strong intensity; (vs)=very strong intensity; (s)=strong intensity; (m)=medium intensity; (w)=weak intensity and (vw)=very weak intensity.

The X-ray powder diffraction pattern shows some very intense peaks, caused by the large particle size of the sample. This sample was slightly ground to reduce the particle size to approximately 1 to 10 μm and to avoid this textural effect. The strongest peak intensities are thus reduced and a few of the small peaks disappear. Crystal form A is still present after grinding.

In yet another embodiment, Form A is a crystalline form of (+)- or (−)-mefloquine hydrochloride, which exhibits a characteristic X-ray powder diffraction pattern with characteristic peaks expressed in d-values (Å), when using small-sized particles of a size distribution of 1 to 10 μm:
11.2 (m), 9.0 (w), 8.30 (vw), 7.4 (vw), 6.8 (vw), 6.3 (w), 6.1 (vw), 6.0 (vw), 5.95 (vw), 5.59 (w), 5.42 (w), 4.91 (vw), 4.74 (w), 4.55 (vw), 4.16 (w), 4.12 (s), 4.03 (w), 3.82 (vvs), 3.75 (w), 3.71 (w), 3.64 (w), 3.55 (w), 3.47 (vw), 3.40 (vw), 3.33 (w), 3.26 (w), 3.11 (vw), 3.04 (vw), 2.94 (vw), 2.75 (Tw), 2.71 (vw), 2.69 (vw), 2.64 15 (w), 2.62 (vw), 2.54 (vw), 2.46 (vw), 2.43 (vw), 2.40 (vw), 2.35 (vw), 2.30 (vw), 2.26 (vw), 2.22 (vw), 2.17 (w), 2.08 (vw), 2.06 (vw), 1.99 (vw), 1.91 (vw) and 1.89 (vw).

In still another preferred embodiment of the present invention, crystalline form A of (+)- or (−)-mefloquine hydrochloride exhibits characteristic X-ray powder diffraction patterns as exhibited in FIG. 1, 2 or 3.

In another preferred embodiment of the present invention, Form A comprises additionally a crystalline form of (+)- or (−)-mefloquine hydrochloride which exhibits characteristic Raman bands, expressed in wave numbers (cm^{-1):} 1030.2 (w) and 85.4 (vs).

Of forms A, B and C, crystalline form C is the least stable form, and transforms to crystalline form B. Crystal form B is also metastable and transforms into the thermodynamically stable crystalline form A. A crystallization process using ethanol/water mixtures can produce only crystalline forms A, B and C. The most likely contaminant in crystalline form A may therefore be crystalline form B. Crystalline form A may contain small amounts of crystalline form B. The content of crystalline form A is preferably at least 70, more preferably at least 80, and most preferably at least 90% by weight, referred to the mixture. Pharmacological properties such as bioavailability are not substantially affected by a certain content of crystalline form B.

Other Forms

It has also been found that the product re-crystallised from a mixture of acetonitrile and methylene chloride (see Carroll et al, supra) yields a mixture of crystalline acetonitrile and methylene chloride solvates. It was surprisingly found that solvates can also be produced with acetone, tetrahydrofuran and methyl ethyl ketone, and that these solvates can be used to produce other crystalline forms of (+)- or (−)-mefloquine hydrochloride, for example crystalline form D, obtainable by de-solvating the methyl ethyl ketone solvate.

Form D

Another aspect of the invention is another, crystalline form, which differs from forms A, B and C, and which can be produced by removing solvent from a methyl ethyl ketone solvate. Form D is a crystalline form of (+)- or (−)-mefloquine hydrochloride which exhibits characteristic Raman bands, expressed in wave numbers (cm⁻¹):
2877 (m), 1601 (s), 1585 (s), 1363 (vs), 1028.2 (w), 320 (m) and 118 (vs).
   Form E

A further aspect of the invention is a crystalline pseudo-polymorph of (+)- or (−)-mefloquine hydrochloride, which exhibits characteristic Raman bands, expressed in wave numbers (cm⁻¹):
1602 (s), 1585 (s),1363 (vs), 322 (m) and 118 (vs).
in the form of the acetone solvate. The content of acetone may be from 0.8 to 1 mol, referred to (+)- or (−)-mefloquine hydrochloride.
   Form F

Yet another aspect of the invention is a crystalline pseudo-polymorph of (+)- or (−)-mefloquine hydrochloride which exhibits characteristic Raman bands, expressed in wave numbers (cm⁻¹):
1601 (s), 1585 (s), 1363 (vs), 323 (m) and 119 (vs);
in the form of the tetrahydrofuran solvate. The content of tetrahydrofuran may be from 0.8 to 1 mol, referred to (+)- or (−)-mefloquine hydrochloride.
   Form G

Still another aspect of the invention is a crystalline pseudo-polymorph of (+)- or (−)-mefloquine hydrochloride which exhibits characteristic Raman bands, expressed in wave numbers (cm⁻¹):
1600 (s), 1585 (s), 1363 (vs), 319 (m) and 118 (vs);
in the form of the methyl ethyl ketone solvate. The content of methyl ethyl ketone may be from 0.8 to 1 mol, referred to (+)- or (−)-mefloquine hydrochloride.

### BREIF DESCRIPTION OF THE DRAWINGS

FIG. 1 is a characteristic X-ray powder diffraction pattern of form A (Synchrotron measurement).

FIG. 2 is a characteristic X-ray powder diffraction pattern of form A (large-sized particles).

FIG. 3 is a characteristic X-ray powder diffraction pattern of form A (small-sized particles).

FIG. 4 is a characteristic X-ray powder diffraction pattern of form B (Synchrotron measurement).

FIG. 5 is a characteristic Raman spectrum of form A [(+)-enantiomer].

FIG. 6 is a characteristic Raman spectrum of form A [(−)-enantiomer].

FIG. 7 is a characteristic Raman spectrum of form B.

FIG. 8 is a characteristic Raman spectrum of form C.

FIG. 9 is a characteristic Raman spectrum of form D.

FIG. 10 is a characteristic Raman spectrum of form E.

FIG. 11 is a characteristic Raman spectrum of form F.

FIG. 12 is a characteristic Raman spectrum of form G.

FIG. 13a is a scanning electron microscope image of form A (cuboid and cubic-like morphology) prepared by crystallization in ethanol/water without seeding.

FIG. 13b is a scanning electron microscope image of form A (cuboid and cubic-like morphology) prepared by crystallization in ethanol/water with seeding.

### DESCRIPTION OF THE INVENTION

The crystalline polymorphic forms A, B and C can have different crystal habits such as cubes, cube-like particles, columns, needles or blade-shaped particles. Thick columns, cuboids, cubes and cube-like habits are preferred, regarding their advantageous handling and processing properties. Cuboids, cubes and cube-like particles are particularly preferred. Mixtures of crystal habits are possible, including those with a predominant part of thick columns, cuboids, cubes and cube-like forms and small parts of needles and/or blade-shaped particles. The particle size may be in the range of 1 to 1000 μm, preferably 10 to 700 μm, and more preferably 20 to 500 μm, referred to the longest edge of morphological form.

Crystalline polymorphic forms A, B, C, D, E, F and G of (+)- or (−)-mefloquine hydrochloride are preferably substantially in the habit of thick columns, cuboids, cubes or cube-like particles, and particularly preferred in the form of cuboids, cubes or cube-like particles.

A preferred aspect of the invention is crystalline polymorphic form A of (+)- or (−)-mefloquine hydrochloride substantially in the habit of thick columns, cuboids, cubes or cube-like particles, and particularly preferred in the form of cuboids, cubes or cube-like particles.

For the preparation of the crystal habits, there may be used crystallisation techniques well known in the art, such as suspension, precipitation, re-crystallisation, evaporation, solvent-like water sorption methods or de-solvation of solvates. Diluted, saturated or super-saturated solutions may be used for crystallisation, with or without seeding with suitable nucleating agents. Temperatures up to 150° C. and preferably up to 100° C. may be applied to form solutions. Cooling to initiate crystallisation and precipitation down to −50° C. and preferably down to −10° C. to 30° C. (room temperature) may be applied. Metastable crystalline forms can be used to prepare solutions or suspensions for the preparation of more stable forms and to achieve higher concentrations in the solutions. Crystal forms such as B, C or mixtures thereof as well as solvates may be used to produce crystalline form A or pseudo-polymorphic forms. Pseudo-polymorphic forms may also be used to prepare crystalline form A.

Suitable solvents are for example alkanols such as ethanol and isopropanol, acetic acid esters such as ethyl acetate and mixtures of said solvents with lower amounts of water.

It has been surprisingly found that water containing solvent mixtures can be used since no classical hydrate formation of (+)- and (−)-mefloquine hydrochloride is observed (the “0.25-hydrate” reported by Karle et al can be explained as residual water in channels within the crystalline lattice). Moreover, it was also surprisingly found that (+)- or (−)-mefloquine hydrochloride show an unusual solubility behaviour in solvent/water mixtures such as ethanol and water. Solubility in a solvent is increased with the addition of certain amounts of water to pure ethanol and solubility decreases with the addition of higher amounts of water, so that solubility is lower than in pure ethanol at a water content of above 50% (v/v). This effect may be used to initiate precipitation and crystallisation by the addition of water to a solution of (+)- or (−)-mefloquine hydrochloride and also to apply seeding techniques using seeds with a desired morphology such as crystalline form A in cubic or cube-like form. However, other non-solvents may be used to initiate precipitation from solution in a solvent such as hydrocarbons (hexane, heptane, cyclohexane and methylcyclohexane) or ethers (t-butyl methyl ether). Stirring of a suspension for a time sufficient to complete formation of crystalline form A is preferably applied, whereby the time needed may be hours to several days, for example 1 hour to 10 days or more preferably 5 hours to 5 days.

A preferred aspect of the invention is a process for the preparation of crystalline form A of (+)- or (−)-mefloquine hydrochloride, comprising dissolution of a solid form other than form A of (+)- or (−)-mefloquine hydrochloride at a temperature from 20° C. to 100° C. in a solvent to form a concentrated solution, cooling the solution to precipitate (+)- or (−)-mefloquine hydrochloride, stirring the suspension for a time sufficient to complete formation of crystalline form A, removing the solvent and drying the solid residue. A solid form other than form A encompasses crystalline form A, which is contaminated with e.g. forms B and/or C, or which has an undesired morphology like needles or blade-shaped particles. The process may be carried out with or without seeding.

The temperature range of the solution may be from 20° C. to 100° C. and preferably from 20° C. to 70° C. Cooling may be carried out continuously or stepwise and cooling rates may be controlled such that the rates are in the range from 0.1° C./h to 5° C./h and preferably from 0.3° C./h to 3° C./h. Cooling may be stopped at a certain lower temperature level and kept at this temperature until crystallisation is completed. The concentration of (+)- or (−)-mefloquine hydrochloride in the solution may be from 60 to 600 mg/ml and preferably from 80 to 450 mg/ml solvent, depending on the dissolution temperature. Suitable solvents are for example ethanol, isopropanol, ethyl acetate or ethanol/water mixtures in a 80:20 volume ratio. Stirring time may be from 1 hour to 5 days. Isolation of the solid may be done by decantation or filtration. Drying is preferably carried out at about room temperature or at a temperature up to 60° C.

Another preferred aspect of the invention is a process for the preparation of crystalline form A of (+)- or (−)-mefloquine hydrochloride, comprising dissolution of a solid form other than form A of (+)- or (−)-mefloquine hydrochloride at a temperature from 20° C. to 100° C. in a solvent to form a concentrated solution, adding a sufficient amount of a non-solvent to precipitate or (−)-mefloquine hydrochloride, stirring the suspension for a time sufficient to complete formation of crystalline form A, removing the solvent and drying the solid residue. Optionally, the solution may be cooled after addition of a non-solvent. Suitable solvents are for example ethanol, isopropanol or ethyl acetate and suitable non-solvents are for example heptane and or preferably water. The amount of non-solvent added may be one half or up to five times, preferably three times, of the volume of solvent used for dissolution. Other conditions as described before may be applied when carrying out this process. A solid form other than form A encompasses crystalline form A, which is contaminated with e.g. forms B and/or C, or which has an undesired morphology like needles or blade-shaped particles. The process may be carried out with or without seeding.

The unusual solution behaviour of (+)- and (−)-mefloquine hydrochloride in mixtures of ethanol and water as mentioned before can also be the basis for the preparation of crystalline form A, starting from the free base (+)- and (−)-mefloquine, formation of the hydrochloride as a first step and adjusting crystallisation conditions regarding concentration of the free base in the ethanol/water mixture, appropriate water content at each step of the crystallisation process, type and time of seeding to obtain the desired morphology, cooling rate, temperature, time of water addition and phase equilibration. This method provides surprisingly a reliable and convenient process for the manufacture of only crystalline form A, especially in an easy to handle morphological form such as thick columns, cuboids, cubes or cube-like forms. The presence of undesired and unstable crystalline forms B and C cannot even be detected in the final product form A.

A further and preferred aspect of the present invention is a process for the preparation of crystalline form A of (+)- or (−)-mefloquine hydrochloride, comprising the steps of:
a) dissolving or suspending substantially water-free (+)- or (−)-mefloquine free base at temperatures from 10 to 80° C. in ethanol,
b) adding aqueous HCl and water at a concentration, that the water content provides insolubility of the formed (+)- or (−)-mefloquine hydrochloride,
c) shaking or stirring the formed suspension and optionally cooling the mixture,
d) storing the mixture after optional cooling under shaking or stirring, and
e) isolating the precipitate and drying the solid residue.

Seeding may be carried out during or after addition of water in step b) with seeds and amounts of seeds as described later.

Substantially water-free means in the context of the invention that the free base contains not more than 5 and preferably not more than 1 percent by weight of water, referred to the free base. The temperature is preferably about room temperature (20 to 30° C). The water content provided in step b) may be such that the water content in the ethanol/water mixture is at least 40 volume percent, preferably in the range from 40 to 90 volume percent and more preferably from 65 to 85 volume percent, generated by the addition of aqueous HCl and water. The amount of added hydrogen chloride is preferably equivalent to a complete formation of (+)- or (−)-mefloquine hydrochloride and an excess of up to 80% of the equimolar amount may be used. Cooling in step c) may mean cooling to room temperature. Storing time in step d) may mean several hours to several days, e.g. from 1 hour to 10 days. The precipitate may be isolatedby decantation or filtration. Selected drying procedures are preferably air drying or drying under vacuum at room temperature or up to 60° C. The concentration of the free base in ethanol may be from 1 00 to 800 mg/ml and more preferably 200 to 600 mg/ml, which depends on the temperature selected in step a).

An especial advantage in the preparation of crystalline form A is to use the effect of increase and decrease of solubility of (+)- or (−)-mefloquine hydrochloride through the addition of water to ethanol. This method provides a robust process for the preparation of crystalline form A of (+)- or (−)-mefloquine hydrochloride in the desired polymorphic form, even under standard conditions, on an industrial scale.

A particularly preferred embodiment of the invention is a process for the preparation of crystalline form A of (+)- or (−)-mefloquine hydrochloride, comprising the steps of:
a) dissolving or suspending substantially water-free (+)- or (−)-mefloquine free base at temperatures from 40 to 80° C. in ethanol,
b) keeping said temperature and addinig aqueous HCl to form (+)- or (−)-mefloquine hydrochloride under shaking or stirring,
c) slowly decreasing the temperature continuously or continuously and stepwise down to about 10° C. to 30° C.,
d) adding water at said decreased temperature to decrease solubility of (+)- or (−)-mefloquine hydrochloride,
e) continuing shaking/stirring at said decreased temperature, and
f) isolating the precipitate and drying the solid residue.

Seeding may be carried out during or after addition of water in step d) with seeds and amounts of seeds as described later.

Substantially water-free means that the free base contains not more than 5 and preferably not more than 1 percent by weight of water, referred to the free base. It may be important to consider this amount of water together with the amount of water added with concentrated aqueous HCl to adjust the total water content to the desired solubility of (+)- or (−)-mefloquine hydrochloride. The temperature is preferably from 50 to 80° C. The amount of the free base is preferably chosen in such a manner that a concentration of from 100 to 800 mg/ml and more preferably 300 to 700 mg/ml of (+)- or (−)-mefloquine hydrochloride is present in step b). The amount depends on the selected temperature.

Addition of aqueous HCl is preferably not carried out at once and addition may be continuous within 1 to 30 minutes, preferably 5 to 20 minutes. It may be advantageous to heat the aqueous HCl to the temperature as applied in step a). It is convenient to use concentrated aqueous HCl (37% m/m) to better control water content. The amount of added hydrogen chloride is preferably equivalent to a complete formation of (+)- or (−)-mefloquine hydrochloride and an excess of up to 80% of the equimolar amount may be used. The amount of water added with or after addition of aqueous HCl is preferably such that the water content in ethanol in step b) is from 20 to 3 and preferably 15 to 5 volume percent. A turbid mixture may be formed after addition of concentrated HCl, since a small part of dissolved (+)- or (−)-mefloquine hydrochloride can precipitate.

The mixture may be shaken/stirred after step b) for a certain time, e.g. 1 minute to 2 hours, and preferably 5 minutes to 1 hour.

Decrease of temperature in step c) may be carried out in two variants. a first variant, the mixture is continuously cooled at a cooling rate of 0.1 to 5, preferably 0.1 to 2, and more preferably 0.2 to 1, K/min, to a temperature of about 10° C. to 30° C., preferably room temperature (20 to 30° C.). In a second variant, the mixture is cooled continuously and stepwise preferably to a temperature, where added seeds are not dissolved in the mixture. Decreasing the temperature depends on the starting temperature; about 5 to 20° C., more preferably 7 to 15° C. and most preferably about 10° C. is sufficient for this purpose.

Seeding with nucleating agents such as crystalline form A in the desired morphology or crystalline seeds with similar morphology may be carried out in adding up to 5, preferably 0.1 to 3, and more preferably 0.5 to 2.5, percent by weight of said form, which may have been previously produced in a separate batch. The most desired morphological form for seeds are cubic or cube-like forms. The amount of seeds is referred to the amount of (+)- or (−)-mefloquine hydrochloride.

Water addition in step d) serves to decrease solubility of (+)- or (−)-mefloquine hydrochloride in the ethanol/water mixture. The amount of added water may be such that the water content in the ethanol/water mixture may be at least 40 volume percent, preferably in the range from 40 to 90 volume percent and more preferably from 65 to 85 volume percent. Water may be added at once, stepwise or continuously. Addition at once may lead to a sudden formation of an undesired precipitate with a too small particle size; a stepwise or continuous addition is preferred therefore. Suitable dosing time for continuous addition may be from 10 to 90 minutes and more preferably from 30 to 60 minutes.

Shaking/stirring is continued after water addition, e.g. for 10 to 180 and preferably 30 to 120 minutes.

After finishing the crystallisation process, the precipitate is filtered off and dried to remove residual ethanol and water. Drying may be carried out in vacuum, at elevated temperatures or in vacuum and at elevated temperatures, but below the decomposition-temperature. Drying temperatures may be from 10 to 70 ° C. and preferably 20 to 50° C.

An especially preferred process of the invention for the preparation of the crystalline form A of (+)- or (−)-mefloquine hydrochloride in form of cubes or cube-like forms comprises the steps of:
a) dissolving or suspending substantially water-free (+)- or (−)-mefloquine free base at temperatures from 65 to 80° C. in absolute ethanol,
b) keeping said temperature and continuously adding within 5 to 20 minutes under shaking or stirring concentrated aqueous HCl such that the water content in the mixture ethanol/water is from 20 to 3 and preferably 15 to 5 volume percent, and a solution of (+)- or (−)-mefloquine hydrochloride in the ethanol/water mixture is formed,
c) continuously decreasing the temperature at a rate of 0.2 to 1 K/min down to about 20° C. to 30° C., or continuously decreasing the temperature in a first step at a rate of 0.2 to 1 K/min 5 to 20° C. lower as in step a, adding 0.5 to 2.5 percent by weight, referred to the amount of (+)- or (−)-mefloquine hydrochloride, crystalline seeds of crystalline form A in cubic or cube-like morphological form, stirring for 15 to 30 minutes, and then continuously decreasing the temperature at a rate of 0.1 to 1 K/min down to about 20° C. to 30° C.,
d) adding water at said decreased temperature over 30 to 60 minutes in such amount that the water content in the mixture ethanol/water is from 65 to 85 volume percent,
e) continuing shaking/stirring for 1 to 2 hours at said decreased temperature, and
f) isolating the precipitate and drying the solid residue.

Still a further aspect of the invention is a process for the manufacture of (+)- or (−)-mefloquine hydrochloride in crystalline form D, comprising
a) treating with or without vacuum a methyl ethyl ketone solvate of (+)- or (−)-mefloquine hydrochloride at temperatures from 20° C. to 100° C., preferably 30° C. to 70° C., until removal of methyl ethyl ketone, or
b) suspending a methyl ethyl ketone solvate of (+)- or (−)-mefloquine hydrochloride in a non-solvent, stirring for a time sufficient to remove methyl ethyl ketone from the solvate to form crystalline form D, isolating and then drying the isolated crystals.

Suitable non-solvents include, for example, n-heptane, t-butyl methyl ether and water. Stirring in step b) and drying may be carried out at temperatures from 20 to 50° C.

Still a further aspect of the invention is a process for the manufacture of (+)- or (−)-mefloquine hydrochloride in form of the solvates with acetone (form E), tetrahydrofuran (form F) or methyl ethyl ketone (form G), comprising
a) dissolving (+)- or (−)-mefloquine hydrochloride in acetone, tetrahydrofuran or methyl ethyl ketone as solvent at temperatures from 40 to 80° C. to form a concentrated, saturated or super-saturated solution, cooling and stirring the cooled suspension for a time period sufficient to form the solvates, isolating and drying the isolated crystals, or
b) suspending (+)- or (−)-mefloquine hydrochloride in acetone or tetrahydrofuran as solvent, stirring the suspension at temperatures from 20 to 35° C. for a time period sufficient to form the solvates, isolating and drying the isolated crystals.

Suitable time periods to form the solvates are for example from 1 h to 100h and preferably from 2h to 80h. Cooling may mean from −10 to 20° C. and preferably −10 to 10° C. Isolation and drying may be carried out carefully, e.g. at room temperature.

The crystalline forms B to G may be used in pharmaceutical compositions and more preferably as intermediates and starting materials to produce the particularly preferred form A, which can be easy processed and handled due to its stability, possibility for preparation by targeted conditions, its suitable morphology and particle size. These outstanding properties renders polymorph form A especially feasible for pharmaceutical application.

Accordingly, this invention is also directed to a pharmaceutical composition comprising the crystalline forms B, C and/or D of (+)- or (−)-mefloquine hydrochloride substantially in the form of thick columns, cuboids, cubes or cube-like particles, and a pharmaceutically acceptable carrier or diluent.

In a preferred embodiment, this invention is also directed to a pharmaceutical composition comprising the crystalline form A of (+)- or (−)-mefloquine hydrochloride and a pharmaceutically acceptable carrier or diluent. Preferably, the pharmaceutical composition contains crystalline form A substantially in the form of thick columns, cuboids, cubes or cube-like particles.

The amount of crystalline forms of (+)- or (−)-mefloquine hydrochloride substantially depends on type of formulation and desired dosages during administration time periods. The amount in an oral formulation may be from 0.1 to 50 mg, preferably from 0.5 to 30 mg, and more preferably from 1 to 15 mg.

Oral formulations may be solid formulations such as capsules, tablets, pills and troches, or liquid formulations such as aqueous suspensions, elixirs and syrups. Solid and liquid formulations encompass also incorporation of the crystalline forms of (+)- or (−)-mefloquine hydrochloride according to the invention into liquid or solid food. Liquids also encompass solutions of form A of (+)- or (−)-mefloquine hydrochloride for parenteral applications such as infusion or injection.

The crystalline forms according to the invention may be directly used as powders (micronized particles), granules, suspensions or solutions, or they may be combined together with other pharmaceutically acceptable ingredients in admixing the components and optionally finely divide them, and then filling capsules, composed for example from hard or soft gelatine, compressing tablets, pills or troches, or suspend or dissolve them in carriers for suspensions, elixirs and syrups. Coatings may be applied after compression to form pills.

Pharmaceutically acceptable ingredients are well known for the various types of formulation and may be for example binders such as natural or synthetic polymers, excipients, lubricants, surfactants, sweetening and flavouring agents, coating materials, preservatives, dyes, thickeners, adjuvants, antimicrobial agents and carriers for the various formulation types.

Examples of binders are gum tragacanth, acacia, starch, gelatine, and biological degradable polymers such as homo- or co-polyesters of dicarboxylic acids, alkylene glycols, polyalkylene glycols and/or aliphatic hydroxyl carboxylic acids; homo- or co-polyamides of dicarboxylic acids, alkylene diamines, and/or aliphatic amino carboxylic acids; corresponding polyester-polyamide-co-polymers, polyanhydrides, polyorthoesters, polyphosphazene and poly-carbonates. The biological degradable polymers may be linear, branched or crosslinked. Specific examples are poly-glycolic acid, poly-lactic acid, and poly-d,l-lactide/glycolide. Other examples for polymers are water-soluble polymers such as polyoxaalkylenes (polyoxaethylene, polyoxapropylene and mixed polymers thereof), poly-acrylamides and hydroxylalkylated polyacrylamides, poly-maleic acid and esters or amides thereof, poly-acrylic acid and esters or amides thereof, polyvinyl alcohol and esters or ethers thereof, polyvinylimidazole, poly-vinylpyrrolidone, and natural polymers like chitosan.

Examples of excipients are phosphates such as dicalcium phosphate. Examples of lubricants are natural or synthetic oils, fats, waxes, or fatty acid salts like magnesium stearate.

Surfactants may be anionic, anionic, amphoteric or neutral. Examples for surfactants are lecithin, phospholipids, octyl sulfate, decyl sulfate, dodecyl sulfate, tetradecyl sulfate, hexadecyl sulfate and octadecyl sulfate, Na oleate or Na caprate, 1-acylaminoethane-2-sulfonic acids, such as 1-octanoylaminoethane-2-sulfonic acid, 1-decanoylaminoethane-2-sulfonic acid, 1-dodecanoylaminoethane-2-sulfonic acid, 1-tetradecanoylaminoethane-2-sulfonic acid, 1-hexadecanoylaminoethane-2-sulfonic acid, and 1-octadecanoylaminoethane-2-sulfonic acid, and taurocholic acid and taurodeoxycholic acid, bile acids and their salts, such as cholic acid, deoxycholic acid and sodium glycocholates, sodium caprate or sodium laurate, sodium oleate, sodium lauryl sulphate, sodium cetyl sulphate, sulfated castor oil and sodium dioctylsulfosuccinate, cocamidopropylbetaine and laurylbetaine, fatty alcohols, cholesterols, glycerol mono- or -distearate, glycerol mono- or -dioleate and glycerol mono- or -dipalmitate, and polyoxyethylene stearate.

Examples of sweetening agents are sucrose, fructose, lactose or aspartame. Examples of flavouring agents are peppermint, oil of wintergreen or fruit flavours like cherry or orange flavour. Examples of coating materials gelatine, wax, shellac, sugar or biological degradable polymers. Examples of preservatives are methyl or propylparabens, sorbic acid, chlorobutanol, phenol and thimerosal. Examples of adjuvants are fragrances. Examples of thickeners are synthetic polymers, fatty acids and fatty acid salts and esters and fatty alcohols. Examples of liquid carriers are water, alcohols such as ethanol, glycerol, propylene glycol, liquid polyethylene glycols, triacetin and oils. Examples for solid carriers are talc, clay, microcrystalline cellulose, silica, alumina and the like.

The formulation according to the invention may also contain isotonic agents, such as sugars, buffers or sodium chloride.

A crystalline form according to the invention may also be formulated as effervescent tablet or powder, which disintegrate in an aqueous environment to provide a drinking solution.

A syrup or elixir may contain the polymorph of the invention, sucrose or fructose as sweetening agent a preservative like methylparaben, a dye and a flavouring agent.

Slow release formulations may also be prepared from a crystalline form according to the invention in order to achieve a controlled release of the active agent in contact with the body fluids in the gastro-intestinal tract, and to provide a substantial constant and effective level of the active agent in the blood plasma. The crystalline forms may be embedded for this purpose in a polymer matrix of a biological degradable polymer, a water-soluble polymer or a mixture of both, and optionally suitable surfactants. Embedding can mean in this context the incorporation of micro-particles in a matrix of polymers. Controlled release formulations are also obtained through encapsulation of dispersed micro-particles or emulsified micro-droplets via known dispersion or emulsion coating technologies.

Crystalline forms of the invention are also useful for administering a combination of therapeutic effective agents to an animal. Such a combination therapy can be carried out in using at least one further therapeutic agent which can be additionally dispersed or dissolved in a formulation.

Crystalline forms of this invention and formulations respectively can be also administered in combination with other therapeutic agents that are effective to treat a given condition to provide a combination therapy.

Crystalline forms and pharmaceutical compositions according to the invention are highly suitable for effective treatment of malaria with reduced side-effects, the treatment of movement and neurodegenerative disorders, for the treatment of inflammatory and autoimmune diseases such as rheumatoid arthritis, osteoarthritis, psoriatic arthritis, psoriasis, Crohn's disease, systemic lupus erythematosis (SLE), ulcerative colitis, chronic obstructive pulmonary disease (COPD) and asthma, as described for both enantiomers previously.

An aspect of the invention is also a therapeutic method for producing an anti-malarial, anti-inflammatory and anti-autoimmune, or anti-neurodegenerative effect in a mammal comprising administering to a mammal in need of such therapy, an effective amount of a crystalline form of (+)-mefloquine hydrochloride according to the invention, or respectively a crystalline form of (−)-mefloquine hydrochloride according to the invention.

Another aspect of the invention is a method of delivering a crystalline form of (+)- or (−)-mefloquine hydrochloride according to the invention to a host, comprising administering to a host an effective amount of a crystalline form according to the invention.

A further aspect of the invention is the use of a crystalline form according to the invention for the manufacture of a medicament useful in the treatment of malaria, in the treatment of movement and neurodegenerative disorders, or in the treatment of inflammatory and autoimmune diseases in an mammal, such as a human; and a crystalline form according to the invention for use in medical therapy.

The following Examples illustrate the invention without limiting the scope.

A) Prepaeation of Crystalline Forms A and D

### EXAMPLE A1

### Preparation of crystalline form A

101 mg of (+)-mefloquine free base are dissolved in 0.35 ml ethanol absolute at room temperature. 0.27 ml 1 M aqueous HCl is added and the mixture is shaken. The mixture is stored for 8 days at room temperature without stirring. Subsequent decantation of the mother liquor and air drying of the solid gives (+)-mefloquine hydrochloride crystalline form A in needle form.

### EXAMPLE A2

### Preparation of Crystalline Form A

100 mg of (+)-mefloquine free base are dissolved in 0.35 ml ethanol absolute at room temperature. 0.03 ml concentrated aqueous HCl (37% m/m) is added and the mixture is shaken. The mixture is stored for 1 day at room temperature without stirring. Subsequent decantation of the mother liquor and air drying of the solid gives (+)-mefloquine hydrochloride crystalline form A in cubic morphology.

### EXAMPLE A3

### Preparation of Crystalline Form A

5.01 g pure (+)-mefloquine free base (residual water<1%) are suspended while stirring in 16.2 ml ethanol absolute at room temperature and heated to 70° C. 1.64 ml concentrated aqueous HCl (37% m/m) are added to the solution at 70° C. over 10 minutes and the mixture is stirred for 1 additional hour. The temperature is lowered at a rate of 0.4 K/min to 25° C. while stirring. At 25° C., 46 ml water are added to the suspension at a dosing rate of 32 ml/h. After water addition the suspension is stirred for 45 additional minutes at room temperature. Subsequent filtration and air drying gives (+)-mefloquine hydrochloride crystalline form A in 30 cubic morphology.

### EXAMPLE A4

### Preparation of Crystalline Form A

5.00 g pure (+)-mefloquine free base (residual water content<1%) are suspended while stirring in 16.2 ml absolute ethanol at room temperature and heated to 70° C. 1.64 ml concentrated aqueous HCl (37% m/m) are added to the solution at 70° C. over 10 minutes and the mixture is stirred for 15 additional minutes. The temperature is lowered at a rate of 0.3 K/min to 60° C. while stirring. At 60° C., 50 mg (+)-mefloquine hydrochloride crystalline form A in cubic morphology are added and the suspension is stirred for 5 minutes at 60° C. The temperature is lowered at a rate of 0.3 K/min to 25° C. while stirring. At 25° C., 46 ml water are added to the suspension at a dosing rate of 84 ml/h. After water addition the suspension is stirred for 10 additional minutes at room temperature. Subsequent filtration and drying for 20 hours under vacuum (10 mbar) at 40° C. gives 5.09 g (+)-mefloquine hydrochloride crystalline form A in cubic morphology.

### EXAMPLE A5

### Preparation of Cystalline Form A

5.01 g pure (+)-mefloquine free base (residual water content<1%) are suspended while stirring in 8.1 ml absolute ethanol at room temperature and heated to 69° C. 1.64 ml concentrated aqueous HCl (37% m/m) are added to the solution at 69° C. over 10 minutes and the mixture is stirred for 20 additional minutes. The temperature is lowered at a rate of 0.7 K/min to 25° C. while stirring. At 25° C., 23 ml water are added to the suspension at a dosing rate of 115 ml/h. After water addition the suspension is stirred for 18 additional minutes at room temperature. Subsequent filtration and air drying gives (+)-mefloquine hydrochloride crystalline form A in cubic morphology.

### EXAMPLE A6

### Preparation of Crystalline Form A

5.01 g pure (+)-mefloquine free base (residual water content<1%) are suspended while stirring in 8.1 ml absolute ethanol at room temperature and heated to 70° C. 1.64 ml concentrated aqueous HCl (37% m/m) are added to the solution at 70° C. over 10 minutes. At 70° C. 23 ml water are added to the suspension at a dosing rate of 92 ml/h. After water addition the suspension is stirred for 5 additional minutes at 70° C. The temperature is lowered at a rate of 0.8 K/min to 23° C. while stirring. The suspension is stirred for 10 additional minutes at 23° C. Subsequent filtration and drying for 16 hours under vacuum (15 mbar) at 40° C. gives (+)-mefloquine hydrochloride crystalline form A in cubic morphology.

### EXAMPLE A7

### Preparation of Crystalline Form A

101 mg of (−)-mefloquine hydrochloride are dissolved in a mixture of 1.4 ml ethanol and water (1:1 v/v) at room temperature. 1.4 ml water are added. The mixture is stirred for 5 days at room temperature. Subsequent filtration and air drying of the solid gives (−)-mefloquine hydrochloride crystalline form A (very fine particles).

### EXAMPLE A8

### Preparation of Crystalline Form D

101 mg of (+)-mefloquine hydrochloride are dissolved in 3.5 ml methyl ethyl ketone at 70° C. The mixture is stored for 4 days at 5° C. Subsequent filtration and air drying of the solid gives (+)-mefloquine hydrochloride in crystalline form D in cubic morphology. (Remark: Form D is an “isomorphic” desolvated solvate of the methyl ethyl ketone solvate).

B) Preparation of Solvates

### EXAMPLE B1

### Preparation of Acetone Solvate

101 mg of (+)-mefloquine hydrochloride are suspended in 5.0 ml acetone at room temperature. The suspension is stirred for 18 hours at room temperature. Subsequent filtration and air drying at room temperature of the solid gives (+)-mefloquine hydrochloride acetone solvate (very fine particles).

### EXAMPLE B2

### Preparation of Acetone Solvate

101 mg of (+)-mefloquine hydrochloride are dissolved in 17 ml acetone at 50° C. The mixture is stored for 2 hours at 5° C. Subsequent filtration and air drying at room temperature of the solid gives (+)-mefloquine hydrochloride acetone solvate (prisms).

### EXAMPLE B3

### Preparation of Tetrahydrofuran Solvate

100 mg of (+)-mefloquine hydrochloride are dissolved in 1.5 ml tetrahydrofuran at 70° C. The mixture is stored for 5 days at 5° C. Subsequent filtration and air drying at room temperature of the solid gives (+)-mefloquine hydrochloride tetrahydrofuran solvate (cubes).

### EXAMPLE B4

### Preparation of Methyl Ethyl Ketone Solvate

301 mg of (+)-mefloquine hydrochloride are dissolved in 9.5 ml methyl ethyl ketone at 75° C. The mixture is stored for 3 days at 5° C. Subsequent air drying at room temperature of the crystals formed gives (+)-mefloquine hydrochloride methyl ethyl ketone solvate (cubes).

C) Preparation of Crystalline Forms B and C

These crystalline forms are prepared according to the new processes of this invention as a comparison with crystalline (+)- and (−)-mefloquine hydrochloride described in Journal of Medicinal Chemistry Vol. 17(2), pages 210 to 219.

### EXAMPLE C1

### Preparation of Crystalline Form B

100 mg of (+)-mefloquine hydrochloride are dissolved in a mixture of 1.4 ml ethanol and water (1:1 v/v) at room temperature. 1.4 ml water are added and the mixture is shaken. The mixture is stored for 23 hours at room temperature without stirring. Subsequent filtration and air drying at room temperature of the solid gives (+)-mefloquine hydrochloride crystalline form B in needle form.

### EXAMPLE C2

### Preparation of Crystalline Form B

100 mg of (+)-mefloquine hydrochloride are dissolved in 2.0 ml ethanol absolute at room temperature. 6.0 ml n-heptane are added and the mixture is stirred for 5 minutes. The mixture is stored for 23 hours at room temperature without stirring. Subsequent filtration and air drying at room temperature of the solid gives (+)-mefloquine hydrochloride crystalline form B in column form.

Crystal form B exhibits a characteristic X-ray powder diffraction pattern with characteristic peaks expressed in d-values (Å), measured with Synchrotron X-ray radiation:
11.3 (s); 9.5 (w); 9.0 (w); 8.3 (w); 6.3 (m); 6.1 (m); 6.0 (w); 5.45(w); 5.25(w); 4.74 (m); 4.20 (m); 4.16 (s); 4.12 (s); 3.81 (vs); 3.77 (w); 3.75 (m); 3.71 (s); 3.64 (w); 3.47 (w); 3.11 (w); 2.75 (w); 2.70 (w); 2.64 (w); 2.62 (w); 2.45 (m); 1.99 (w); and 1.95 (w)

Crystal form B exhibits characteristic Raman bands, expressed in wave numbers (cm⁻¹):
1026.1 (w); 87.4 (vs).

### EXAMPLE C3

### Preparation of Crystalline Form C

300 mg of (+)-mefloquine hydrochloride are dissolved in 4.5 ml absolute ethanol at room temperature. 30 ml n-heptane are added. The mixture is stirred for 0.5 hours at room temperature. Subsequent filtration and air drying at room temperature of the solid gives (+)-mefloquine hydrochloride crystalline form C in columns and blade shaped particles.

### EXAMPLE C4

### Preparation of Crystalline Form C

101 mg of (+)-mefloquine free base are dissolved in 0.35 ml ethanol absolute at room temperature. 10 ml gaseous HCl are added. The suspension is stored for 1.5 hours at room temperature without stirring. Subsequent filtration and air drying at room temperature of the solid gives (+)-mefloquine hydrochloride crystalline form C in cubic morphology.

### EXAMPLE C5

### Preparation of Crystalline Form C

5.01 g pure (+)-mefloquine free base (residual water content <1%) are suspended while stirring in 16.2 ml ethanol absolute at room temperature and heated to 70° C. 1.64 ml concentrated aqueous HCl (37% m/m) are added to the solution at 70° C. over 10 minutes and the solution is stirred for 5 additional minutes. The temperature is lowered at a rate of 1 K/min to 55° C. while stirring. Subsequent filtration of a small sample and air drying at room temperature gives (+)-mefloquine hydrochloride crystalline form C in cubic morphology.

Crystal form C exhibits characteristic Raman bands, expressed in wave numbers (cm⁻¹):
2962 (s); 2958 (s); 1026.2 (w) and 88.3 (vs).

### Experimental

Powder X-ray Diffraction (PXRD): PXRD is performed on a Philips 1710 powder X-ray diffractometer using CuK_{α} radiation. D-spacings are calculated from the 2θ values using the wavelength of 1.54060 Å. Generally, 2θ values are within an error of ±0.1-0.2°. The experimental error on the d-spacing values is therefore dependent on the peak location.

The synchrotron radiation X-ray diffraction is performed according to the method in Material Science Forum Vols. 321-324 (2000), pp. 212 to 217. The sample is loaded into a 1.0 mm diameter glass capillary to a depth of approximately 3 cm. Data collection takes place on station 2.3 of the SRS at Daresbury Laboratory. The wavelength of X-ray used is 1.300 Å (calibrated using a silicon standard), and the beam size is 1.0×10 mm ². Collected data are re-calculated to CuK_{α} radiation of 1.54060 Å.

Raman spectroscopy: FT-Raman spectra are recorded on a Bruker RFS 100 FT-Raman system with a near infrared Nd:YAG laser operating at 1064 nmm and a liquid nitrogen-cooled germanium detector. For each sample, 64 scans with a resolution of 2 cm⁻¹ are accumulated. Generally, 100 mW laser power is used.

1-24. (canceled)

25. A (+)- or (−)-erythro-mefloquine hydrochloride having one or more of the following characteristics:
a) (+)- or (−)-erythro-mefloquine hydrochloride in a crystalline form which exhibits a characteristic X-ray powder diffraction pattern with peaks expressed in d-values (Å) of: 5.95 (s) and 4.02 (w);
b) (+)- or (−)-erythro-Mefloquine hydrochloride comprising particles having a size distribution of 30 to 150 μm, in a crystalline form which exhibits a characteristic X-ray powder diffraction pattern with peaks expressed in d-values (Å) of:
22.3 (vw), 11.2 (vs), 9.0 (w), 8.2 (vw), 7.4 (vw), 6.8 (vw), 6.5 (vw), 6.3 (vw), 6.1 (vw), 6.0 (vw), 5.94 (vw), 5.61 (m), 5.42 (w), 4.89 (vw), 4.74 (w), 4.54 (w), 4.12 (s), 4.02 (w), 3.81 (vvs), 3.74 (vs), 3.70 (vw), 3.64 (w), 3.55 (w), 3.47, (vw), 3.40 (vw), 3.34 (vw), 3.31 (vw), 3.26 (vs), 3.11 (vw), 3.04 (w), 2.97 (vw), 2.94 (vw), 2.81 (vw), 2.75 (m), 2.71 (w), 2.69 (w), 2.64 (w), 2.62 (w), 2.54 (vw), 2.46 (vw), 2.43 (vw), 2.40 (vw), 2.35 (vw), 2.30 (vw), 2.27 (vw), 2.24 (vw), 2.22 (vw), 2.17 (vs), 2.08 (vw), 2.06 (vw), 2.04 (vw), 1.94 (w), 1.91 (vw) and 1.88 (vw);
c) (+)- or (−)-erythro-mefloquine hydrochloride comprising particles having a size distribution of 1 to 10 μm, in a crystalline form which exhibits a characteristic X-ray powder diffraction pattern with peaks expressed in d-values (Å) of:
11.2 (m), 9.0 (w), 8.30 (vw), 7.4 (vw), 6.8 (vw), 6.3 (w), 6.1 (vw), 6.0 (vw), 5.95 (vw), 5.59 (w), 5.42 (w), 4.91 (vw), 4.74 (w), 4.55 (vw), 4.16 (w), 4.12 (s), 4.03 (w), 3.82 (vvs), 3.75 (w), 3.71 (w), 3.64 (w), 3.55 (w), 3.47 (vw), 3.40 (vw), 3.33 (w), 3.26 (w), 3.11 (vw), 3.04 (vw), 2.94 (vw), 2.75 (w), 2.71 (vw), 2.69 (vw), 2.64 (w), 2.62 (vw), 2.54 (vw), 2.46 (vw), 2.43 (vw), 2.40 (vw), 2.35 (vw), 2.30 (vw), 2.26 (vw), 2.22 (vw), 2.17 (w), 2.08 (vw), 2.06 (vw), 1.99 (vw), 1.91 (vw) and 1.89 (vw);
d) (+)- or (−)-erythro-mefloquine hydrochloride in a crystalline form which exhibits characteristic Raman bands, expressed in wave numbers (cm⁻¹), of:
1030.2 (w) and 85.4 (vs);
e) (+)- or (−)-erythro-mefloquine hydrochloride in a crystalline form which exhibits characteristic Raman bands, expressed in wave numbers (cm⁻¹), of:
2877 (m), 1601 (s), 1585 (s), 1363 (vs), 1028.2 (w), 320 (m) and 118 (vs);
f) (+)- or (−)-erythro-mefloquine hydrochloride which, as an acetone solvate, is in the form of a crystalline pseudo-polymorph which exhibits characteristic Raman bands, expressed in wave numbers (cm⁻¹) of:
1602 (s), 1585 (s), 1363 (vs), 322 (m) and 118 (vs);
g) (+)- or (−)-erythro-mefloquine hydrochloride which, as a tetrahydrofuran solvate, is in the form of a crystalline pseudo-polymorph which exhibits characteristic Raman bands, expressed in wave numbers (cm⁻¹), of:
1601 (s), 1585 (s), 1363 (vs), 323 (m) and 119 (vs);
h) (+)- or (−)-erythro-mefloquine hydrochloride which, as a methyl ethyl ketone solvate, exhibits characteristic Raman bands, expressed in wave numbers )(cm⁻¹), of:
1600 (s), 1585 (s), 1363 (vs), 319 (m) and 118 (vs); and
i) (+)- or (−)-erythro-mefloquine hydrochloride, which is substantially in the form of thick columns, cuboids, cubes or cube-like particles.

26. The (+)- or (−)-ethro-mefloquine hydrochloride, according to claim 25, in a crystalline form which exhibits a characteristic X-ray powder diffraction pattern with peaks expressed in d-values (Å) of: 5.95 (s) and 4.02 (w).

27. The (+)- or (−)-erythro-mefloquine hydrochloride according to claim 26, wherein the pattern also has peaks, expressed in d-values (Å), of:
11.2 (vs), 9.0 (s), 7.4 (w), 6.8 (w), 6.3 (s), 6.1 (m), 6.0 (m), 5.95 (s), 5.58 (m), 5.42 (m), 4.91 (m), 4.87 (w), 4.47 (s), 4.55 (w), 4.16 (vs), 4.12 (s), 4.10 (s), 4.02 (w), 3.82 (vs), 3.77 (w), 3.74 (s), 3.71 (vs), 3.64 (m), 3.47 (w), 3.40 (w), 3.33 (w), 3.31 (m), 3.27 (w), 3.25 (w), 3.11 (m), 3.04 (m), 2.94 (m), 2.92 (w), 2.75 (w), 2.70 (m), 2.68 (w), 2.64 (m), 2.62 (m), 2.54 (w), 2.45 (w), 2.39 (w), 2.35 (w), 2.30 (w), 2.29 (w), 2.25 (w), 2.22 (w), 2.18 (w), 2.17 (w), 2.08 (w), 1.99 (m), 1.95 (w), 1.91 (w), and 1.88 (w).

28. The (+)- or (−)-erythro-mefloquine hydrochloride, according to claim 25, comprising particles having a size distribution of 30 to 150 μm, in a crystalline form which exhibits a characteristic X-ray powder diffraction pattern with peaks expressed in d-values (Å) of:
22.3 (vw), 11.2 (vs), 9.0 (w), 8.2 (vw), 7.4 (vw), 6.8 (vw), 6.5 (vw), 6.3 (vw), 6.1 (vw), 6.0 (vw), 5.94 (vw), 5.61 (m), 5.42 (w), 4.89 (vw), 4.74 (w), 4.54 (w), 4.12 (s), 4.02 (w), 3.81 (vvs), 3.74 (vs), 3.70 (vw), 3.64 (w), 3.55 (w), 3,47, (vw), 3.40 (vw), 3.34 (vw), 3.31 (vw), 3.26 (vs), 3.11 (vw), 3.04 (w), 2.97 (vw), 2.94 (vw), 2.81 (vw), 2.75 (m), 2.71 (w), 2.69 (w), 2.64 (w), 2.62 (w), 2.54 (vw), 2.46 (vw), 2.43 (vw), 2.40 (vw), 2.35 (vw), 2.30 (vw), 2.27 (vw), 2.24 (vw), 2.22 (vw), 2.17 (vs), 2.08 (vw), 2.06 (vw), 2.04 (vw), 1.94 (w), 1.91 (vw) and 1.88 (vw).

29. The (+)- or (−)-erythro-mefloquine hydrochloride, according to claim 25, comprising particles having a size distribution of 1 to 10 μm, in a crystalline form which exhibits a characteristic X-ray powder diffraction pattern with peaks expressed in d-values (Å) of:
11.2 (m), 9.0 (w), 8.30 (vw), 7.4 (vw), 6.8 (vw), 6.3 (w), 6.1 (vw), 6.0 (vw), 5.95 (vw), 5.59 (w), 5.42 (w), 4.91 (vw), 4.74 (w), 4.55 (vw), 4.16 (w), 4.12 (s), 4.03 (w), 3.82 (vvs), 3.75 (w), 3.71 (w), 3.64 (w), 3.55 (w), 3.47 (vw), 3.40 (vw), 3.33 (w), 3.26 (w), 3.11 (vw), 3.04 (vw), 2.94 (vw), 2.75 (w), 2.71 (vw), 2.69 (vw), 2.64 (w), 2.62 (vw), 2.54 (vw), 2.46 (vw), 2.43 (vw), 2.40 (vw), 2.35 (vw), 2.30 (vw), 2.26 (vw), 2.22 (vw), 2.17 (w), 2.08 (vw), 2.06 (vw), 1.99 (vw), 1.91 (vw) and 1.89 (vw).

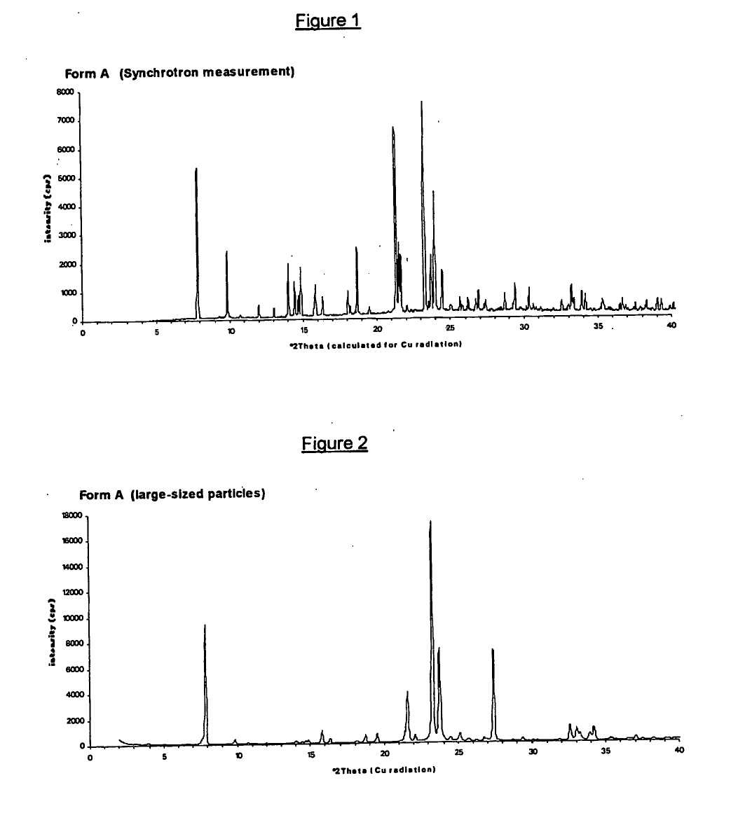

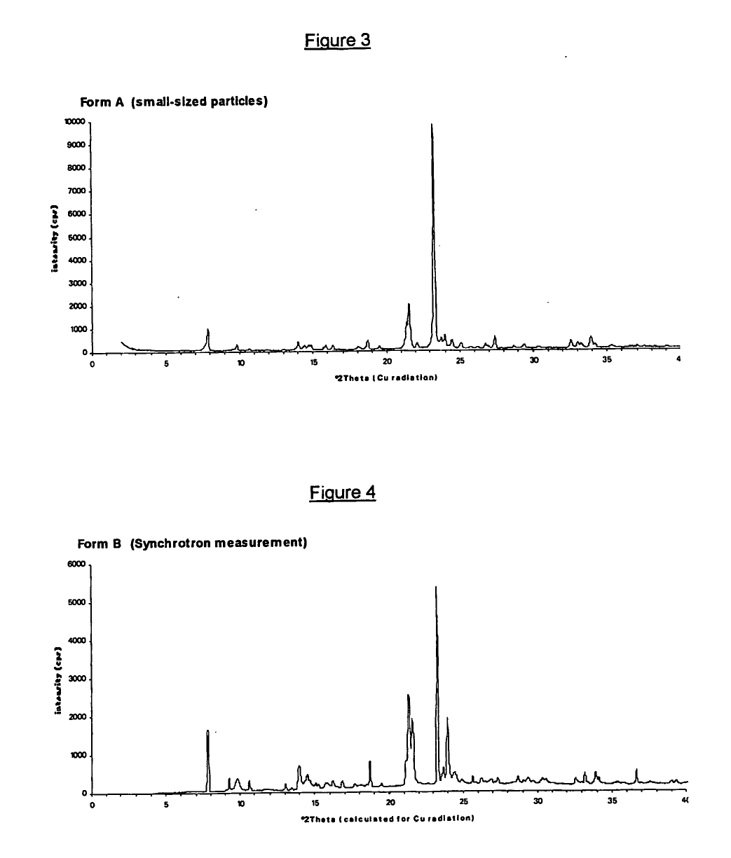

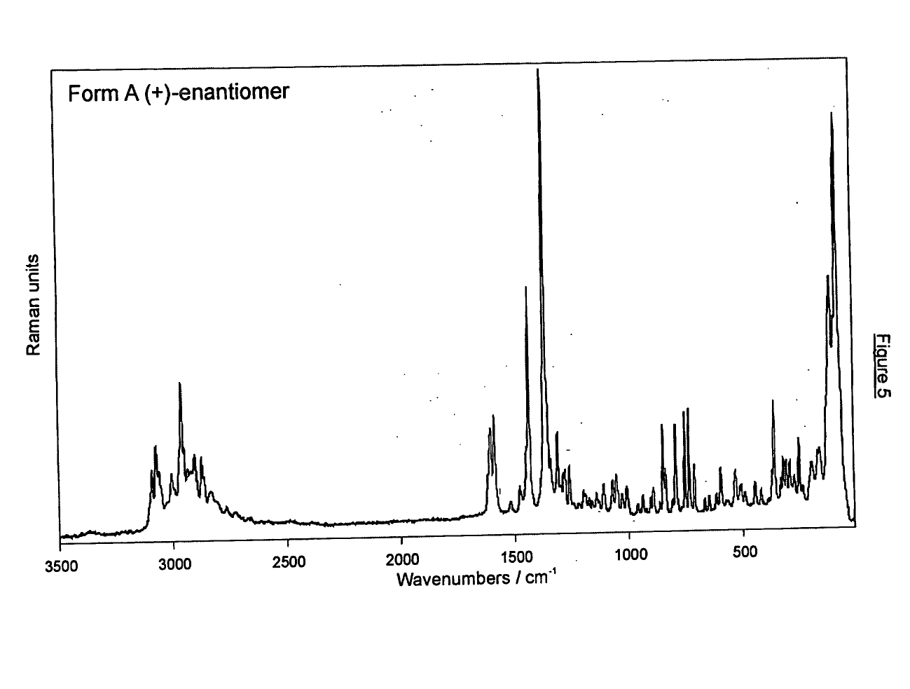

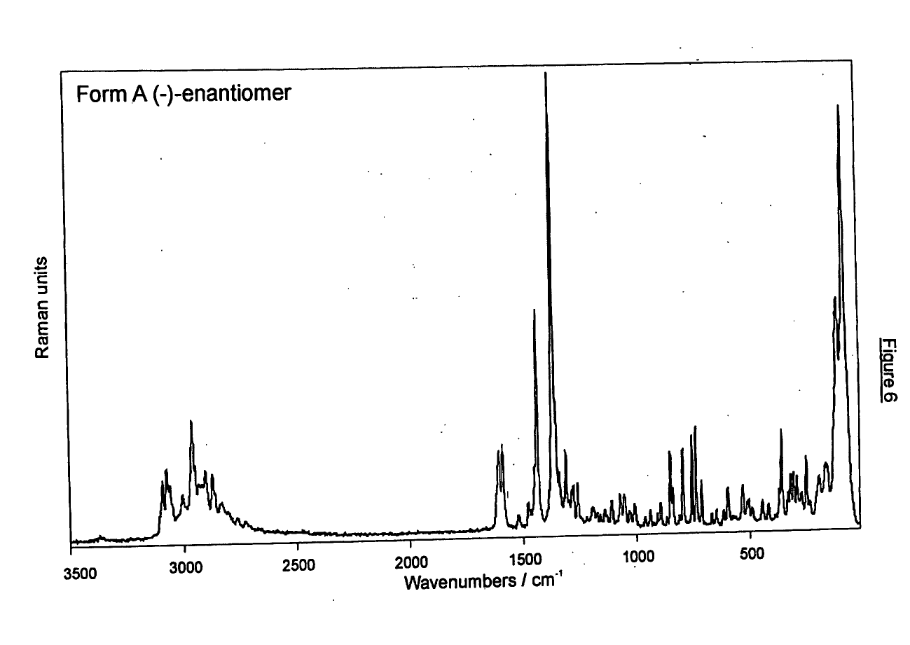

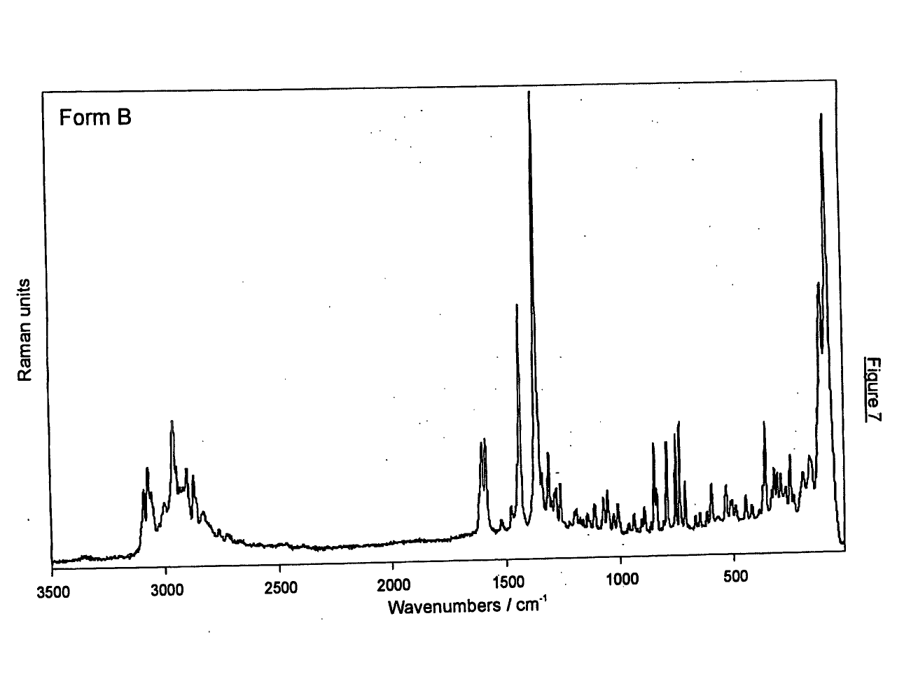

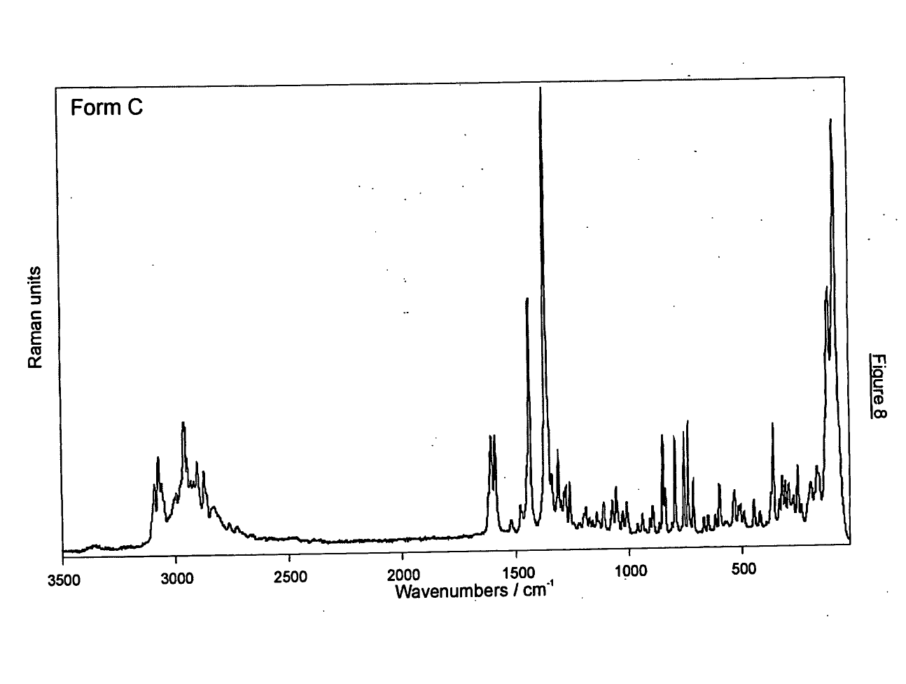

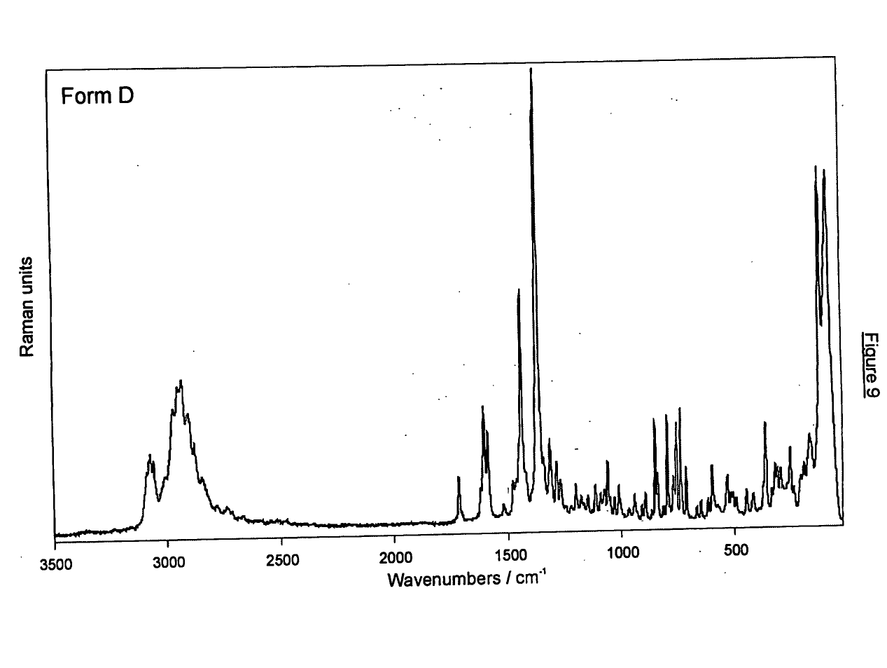

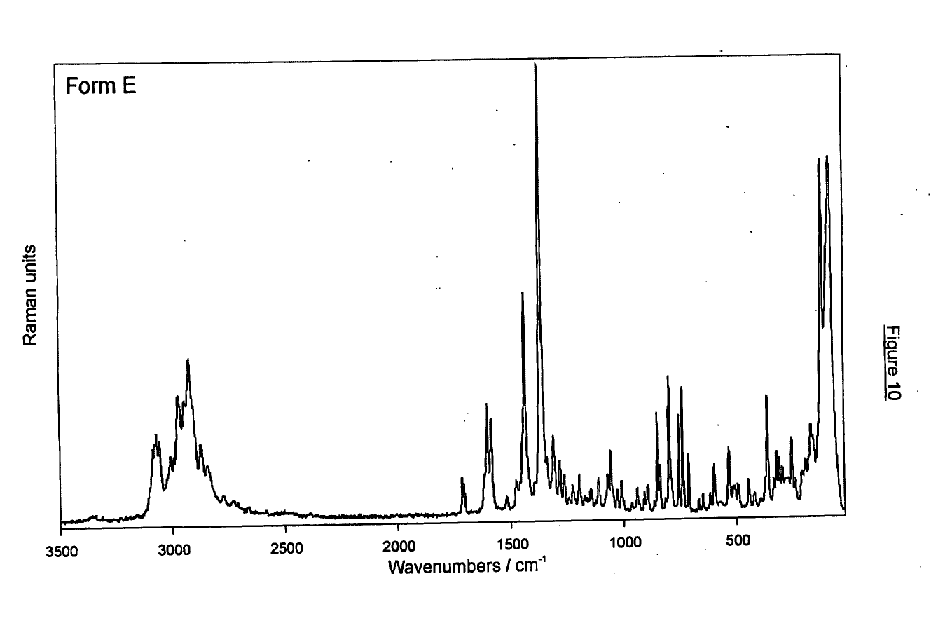

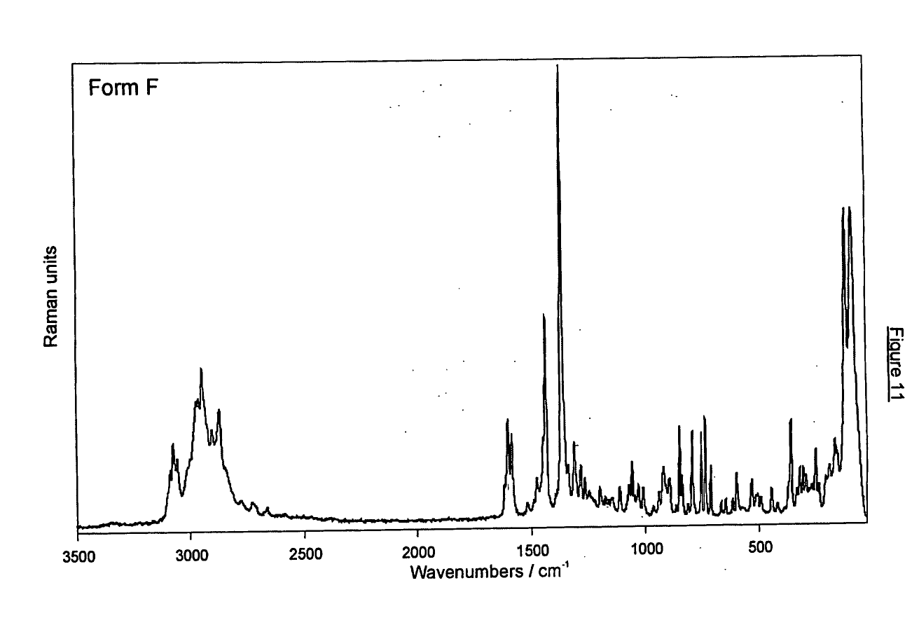

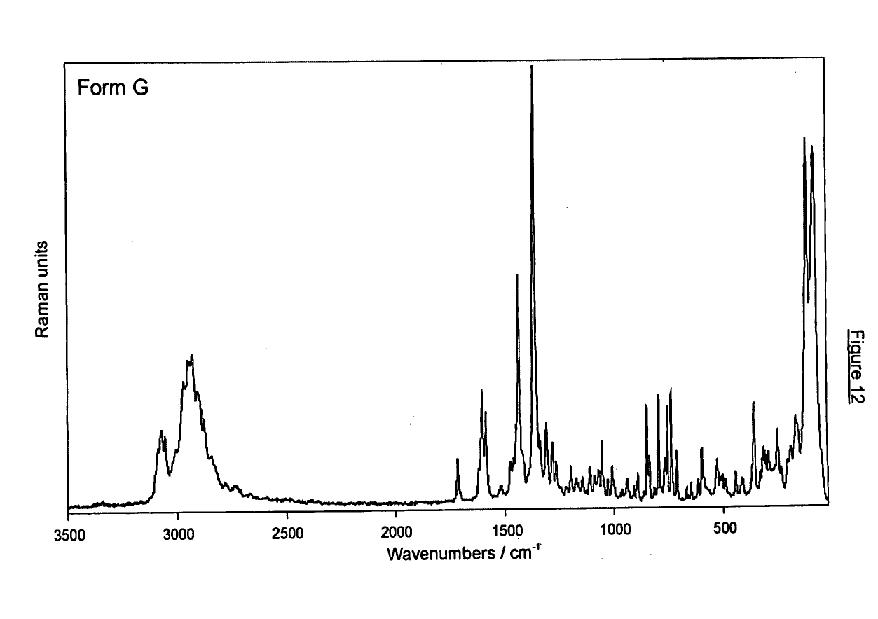

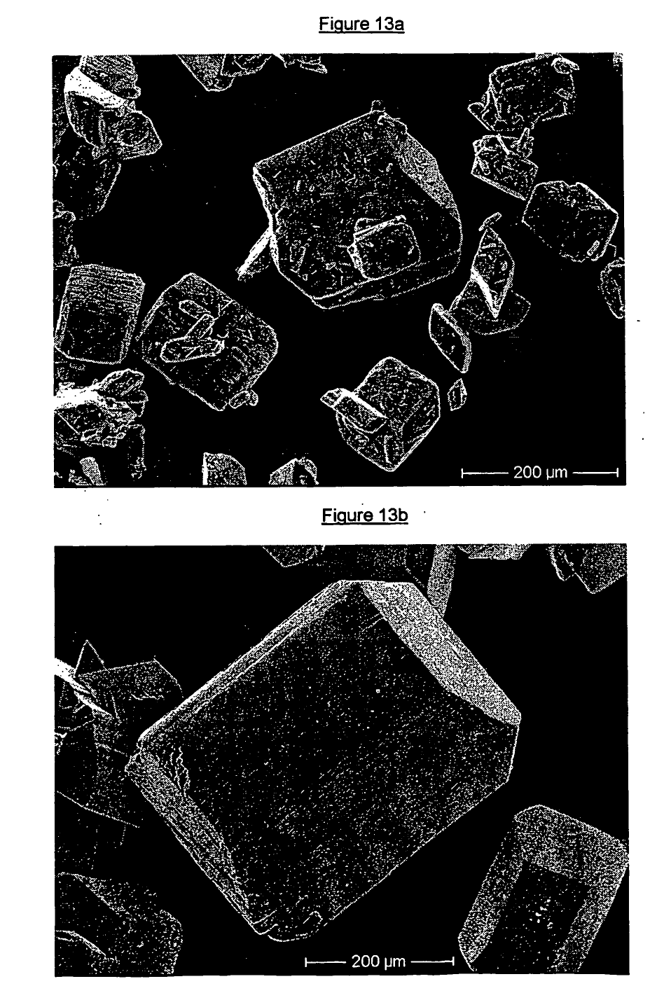

30. The (+)- or (−)-erythro-mefloquine hydrochloride according to claim 25, which exhibits a characteristic X-ray powder diffraction pattern as exhibited in any of FIGS. 1, 2 and 3.

31. The (+)- or (−)-erythro-mefloquine hydrochloride, according to claim 25, in a crystalline form which exhibits characteristic Raman bands, expressed in wave numbers (cm⁻¹) of:
1030.2 (w) and 85.4 (vs).

32. The (+)- or (−)-erythro-mefloquine hydrochloride according to claim 25, in a crystalline form which exhibits characteristic Raman bands, expressed in wave numbers (cm⁻¹), of:
2877 (m), 1601 (s), 1585 (s), 1363 (vs), 1028.2 (w), 320 (m) and 118 (vs).

33. The (+)- or (−)-erythro-mefloquine hydrochloride, according to claim 25 which, as an acetone solvate, is in the form of a crystalline pseudo-polymorph which exhibits characteristic Raman bands, expressed in wave numbers (cm⁻¹) of:
1602 (s), 1585 (s), 1363 (vs), 322 (m) and 118 (vs).

34. The (+)- or (−)-erythro-mefloquine hydrochloride, according to claim 25 which, as a tetrahydrofuran solvate, is in the form of a crystalline pseudo-polymorph which exhibits characteristic Raman bands, expressed in wave numbers (cm⁻¹), of:
1601 (s), 1585 (s), 1363 (vs), 323 (m) and 119 (vs).

35. The (+)- or (−)-erythro-mefloquine hydmchloride according to claim 25, which, as a methyl ethyl ketone solvate, exhibits characteristic Raman bands, expressed in wave numbers (cm⁻¹), of:
1600 (s), 1585 (s), 1363 (vs), 319 (m) and 118 (vs).

36. The (+)- or (−)-erythro-mefloquine hydrochloride according to claims 25, which is substantially in the form of thick columns, cuboids, cubes or cube-like particles.

37. The (+)- or (−)-erythro-mefloquine hydrochloride according to claim 36, in crystalline form B or C.

38. A process for the preparation of a (+)- or (−)-erythro-mefloquine hydrochloride having one or more of the following characteristics:
a) (+)- or (−)-erythro-mefloquine hydrochloride in a crystalline form which exhibits a characteristic X-ray powder diffraction pattern with peaks expressed in d-values (Å) of: 5.95 (s) and 4.02 (w);
b) (+)- or (−)-erythro-mefloquine hydrochloride comprising particles having a size distribution of 30 to 150 μm, in a crystalline form which exhibits a characteristic X-ray powder diffraction pattern with peaks expressed in d-values (Å) of:
22.3 (vw), 11.2 (vs), 9.0 (w), 8.2 (vw), 7.4 (vw), 6.8 (vw), 6.5 (vw), 6.3 (vw), 6.1 (vw), 6.0 (vw), 5.94 (vw), 5.61 (m), 5.42 (w), 4.89 (vw), 4.74 (w), 4.54 (w), 4.12 (s), 4.02 (w), 3.81 (vvs), 3.74 (vs), 3.70 (vw), 3.64 (w), 3.55 (w), 3.47, (vw), 3.40 (vw), 3.34 (vw), 3.31 (vw), 3.26 (vs), 3.11 (vw), 3.04 (w), 2.97 (vw), 2.94 (vw), 2.81 (vw), 2.75 (m), 2.71 (w), 2.69 (w), 2.64 (w), 2.62 (w), 2.54 (vw), 2.46 (vw), 2.43 (vw), 2.40 (vw), 2.35 (vw), 2.30 (vw), 2.27 (vw), 2.24 (vw), 2.22 (vw), 2.17 (vs), 2.08 (vw), 2.06 (vw), 2.04 (vw), 1.94 (w), 1.91 (vw) and 1.88 (vw);
c) (+)- or (−)-erythro-mefloquine hydrochloride comprising particles having a size distribution of 1 to 10 μm, in a crystalline form which exhibits a characteristic X-ray powder diffraction pattern with peaks expressed in d-values (Å) of:
11.2 (m), 9.0 (w), 8.30 (vw), 7.4 (vw), 6.8 (vw), 6.3 (w), 6.1 (vw), 6.0 (vw), 5.95 (vw), 5.59 (w), 5.42 (w), 4.91 (vw), 4.74 (w), 4.55 (vw), 4.16 (w), 4.12 (s), 4.03 (w), 3.82 (vvs), 3.75 (w), 3.71 (w), 3.64 (w), 3.55 (w), 3.47 (vw), 3.40 (vw), 3.33 (w), 3.26 (w), 3.11 (vw), 3.04 (vw), 2.94 (vw), 2.75 (w), 2.71 (vw), 2.69 (vw), 2.64 (w), 2.62 (vw), 2.54 (vw), 2.46 (vw), 2.43 (vw), 2.40 (vw), 2.35 (vw), 2.30 (vw), 2.26 (vw), 2.22 (vw), 2.17 (w), 2.08 (vw), 2.06 (vw), 1.99 (vw), 1.91 (vw) and 1.89 (vw); and
d) (+)- or (−)-erythro-mefloquine hydrochloride in a crystalline form which exhibits characteristic Raman bands, expressed in wave numbers (cm⁻¹), of:
1030.2 (w) and 85.4 (vs);
wherein said process comprises either:
i) dissolution of another solid form of (+)- or (−)-erythro-mefloquine hydrochloride at a temperature from 20° C. to 100° C. in a solvent, to form a concentrated solution, optionally seeding and cooling the solution to precipitate (+)- or (−erythro-mefloquine hydrochloride, stirring the suspension for a time sufficient to complete formation of the desired crystalline form, removing the solvent, and drying the solid residue, or ii) dissolution of another solid form of (+)- or (−)-erythro-mefloquine hydrochloride at a temperature from 20° C. to 100° C. in a solvent, to form a concentrated solution, optionally seeding and adding a sufficient amount of a non-solvent to precipitate (+)- or (−)-erythro-mefloquine hydrochloride, stirring the suspension for a time sufficient to complete formation of the desired crystalline form, removing the solvent, and drying the solid residue.

39. A process for the preparation of a crystalline form of (+)- or (−)-erythro-mefloquine hydrochloride, comprising the steps of:
a) dissolving or suspending substantially water-free (+)- or (−)-erythro-mefloquine free base at a temperature from 10 to 80° C. in ethanol,
b) adding aqueous HCl and water at a concentration, such that the formed (+)- or (−)-erythro-mefloquine hydrochloride is insoluble,
c) shaking or stirring the resultant suspension and optionally also cooling it, and
d) isolating the precipitate and drying the solid residue.

40. The process, according to claim 39, comprising the steps of:
a) dissolving or suspending substantially water-free (+)- or (−)-erythro-mefloquine free base at a temperature from 40 to 80° C. in ethanol,
b) maintahng the temperature and adding aqueous HCl to form (+)- or (−)-erythro-mefloquine hydrochloride under shaking or stirring,
c) slowly decreasing the temperature continuously or continuously and stepwise down to about 10° C. to 30° C.,
d) adding water at the decreased temperature to reduce solubility of (+)- or (−)-erythro-mefloquine hydrochloride,
e) shaking/stiring at the decreased temperature, and
f) isolating the precipitate and drying the solid residue.

41. The process according to claim 39, for the preparation of a (+)- or (−)-erythro-mefloquine hydrochloride in the form of cubes or cube-like forms, having one or more of the following characteristics:
a) (+)- or (−)-erythro-mefloquine hydrochloride in a crystalline form which exhibits a characteristic X-ray powder diffraction pattern with peaks expressed in d-values (Å) of: 5.95 (s) and 4.02 (w);
b) (+)- or (−)-erythro-mefloquine hydrochloride comprising particles having a size distribution of 30 to 150 μm, in a crystalline form which exhibits a characteristic X-ray powder diffraction pattern with peaks expressed in d-values (Å) of:
22.3 (vw), 11.2 (vs), 9.0 (w), 8.2 (vw), 7.4 (vw), 6.8 (vw), 6.5 (vw), 6.3 (vw), 6.1 (vw), 6.0 (vw), 5.94 (vw), 5.61 (m), 5.42 (w), 4.89 (vw), 4.74 (w), 4.54 (w), 4.12 (s), 4.02 (w), 3.81 (ws), 3.74 (vs), 3.70 (vw), 3.64 (w), 3.55 (w), 3.47, (vw), 3.40 (vw), 3.34 (vw), 3.31 (vw), 3.26 (vs), 3.11 (vw), 3.04 (w), 2.97 (vw), 2.94 (vw), 2.81 (vw), 2.75 (m), 2.71 (w), 2.69 (w), 2.64 (w), 2.62 (w), 2.54 (vw), 2.46 (vw), 2.43 (vw), 2.40 (vw), 2.35 (vw), 2.30 (vw), 2.27 (vW), 2.24 (vw), 2.22 (vw), 2.17 (vs), 2.08 (vw), 2.06 (vw), 2.04 (vw), 1.94 (w), 1.91 (vw) anad 1.88 (vw);
c) (+)- or (−)-erythro-mefloquine hydrochloride comprising particles having a size distribution of 1 to 10 μm, in a crystalline form which exhibits a characteristic X-ray powder diffraction pattern with peaks expressed in d-values (Å) of:
11.2 (m), 9.0 (w), 8.30 (vw), 7.4 (vw), 6.8 (vw), 6.3 (w), 6.1 (vw), 6.0 (vw), 5.95 (vw), 5.59 (w), 5.42 (w), 4.91 (vw), 4.74 (w), 4.55 (vw), 4.16 (w), 4.12 (s), 4.03 (w), 3.82 (vvs), 3.75 (w), 3.71 (w), 3.64 (w), 3.55 (w), 3.47 (vw), 3.40 (vw), 3.33 (w), 3.26 (w), 3.11 (vw), 3.04 (vw), 2.94 (vw), 2.75 (w), 2.71 (vw), 2.69 (vw), 2.64 (w), 2.62 (vw), 2.54 (vw), 2.46 (vw), 2.43 (vw), 2.40 (vw), 2.35 (vw), 2.30 (vw), 2.26 (vw), 2.22 (vw), 2.17 (w), 2.08 (vw), 2.06 (vw), 1.99 (vw), 1.91 (vw) and 1.89 (vw); and
d) (+)- or (−)-erythro-mefloquine hydrochloride in a crystalline form which exhibits characteristic Raman bands, expressed in wave numbers (cm⁻¹), of:
1030.2 (w) and 85.4 (vs);
wherein said process comprises the steps of:
a) dissolving or suspending substantially water-free (+)- or (−)-erythro-mefloquine free base at a temperature from 65 to 80° C. in absolute ethanol,
b) maintaining the temperature and continuously adding within 5 to 20 minutes under shaking or stirring concentrated aqueous HCl such that the water content in the ethanol/water mixture is from 20 to 3 and preferably 15 to 5 volume percent, to form a solution of (+)- or (−)-erythro-mefloquine hydrochloride in ethanol/water,
c) continuously decreasing the temperature at a rate of 0.2 to 1 K/min down to about 20° C. to 30° C., or continuously decreasing the temperature in a first step at a rate of 0.2 to 1 K/min 5 to 20° C. lower as in step a), adding 0.5 to 2.5 percent by weight, referred to the amount of (+)- or (−)-erythro-mefloquine hydrochloride, of crystal seeds of the mefloquine hydrochloride according to any of claims 1 to 6, in cubic or cube-like morphological form, stirring 15 to 30 minutes, and then continuously decreasing the temperature at a rate of 0.1 to 1 K/min down to about 20° C. to 30° C.,
d) adding water at the decreased temperature over 30 to 60 minutes in such amount that the water content in the ethanol/water mixture is from 65 to 85 volume percent,
e) continuing shaking/stirring for 1 to 2 hours at the decreased temperature, and
f) isolating the precipitate and drying the solid residue.

42. The process according to claim 39, which comprises storing the mixture between steps (d) and (e).

43. A process for the manufacture of (+)- or (−)-erythro-mefloquine hydrochloride in a crystalline form which exhibits characteristic Raman bands, expressed in wave numbers (cm⁻¹), of:
2877 (m), 1601 (s), 1585 (s), 1363 (vs), 1028.2 (w), 320 (m) and 118 (vs), wherein said process comprises the steps of:
a) treating with or without vacuum a methyl ethyl ketone solvate of (+)- or (−)-erythro-mefloquine hydrochloride at a temperature from 20° C. to 100° C., preferably 30° C. to 70° C., to removel the methyl ethyl ketone, or
b) suspending a methyl ethyl ketone solvate of (+)- or (−)-erythro-mefloquine hydrochloride in a non-solvent, stirring for a time sufficient to remove methyl ethyl ketone from the solvate, and isolating and then drying the crystals.

44. A process for the manufacture of (+)- or (−)-erythro-mefloquine hydrochloride comprising the steps of:
a) dissolving (+)- or (−)-erythro-mefloquine hydrochloride in acetone, tetrahydrofuran or methyl ethyl ketone at a temperature from 40 to 80° C. to form a concentrated, saturated or super-saturated solution, cooling and stirring the cooled suspension for a time period sufficient to form the solvate, and isolating and drying the crystals, or
b) suspending (+)- or (−)-erythro-mefloquine hydrochloride in acetone or tetrahydrofran, stirring the suspension at a temperature from 20 to 35° C. for a time sufficient to form the solvate, and isolating and drying the crystals,
and wherein the (+)- or (−)-eythro-mefloquine hydrochloride has one or more of the following characteristics:
i) as an acetone solvate, is in the form of a crystalline pseudo-polymorph which exhibits characteristic Raman bands, expressed in wave numbers (cm⁻¹) of:
1602 (s), 1585 (s), 1363 (vs), 322 (m) and 118 (vs);
ii) as a tetrahydrofuran solvate, is in the form of a crystalline pseudo-polymorph which exhibits characeistic Raman bands, expressed in wave numbers (cm⁻¹), of:
1601 (s), 1585 (s), 1363 (vs), 323 (m) and
iii) as a methyl ethyl ketone solvate, exhibits characteristic Raman bands, expressed in wave numbers (cm⁻¹), of:
1600 (s), 1585 (s), 1363 (vs), 319 (m) and 118 (vs).

45. A pharmaceutical composition comprising a pharmaceutically acceptable carrier and a (+)- or (−)-erythro-mefloquine hydrochloride having one or more of the following characteristics:
a) (+)- or (−)-erythro-mefloquine hydrochloride in a crystalline form which exhibits a characteristic X-ray powder diffraction pattern with peaks expressed in d-values (Å) of: 5.95 (s) and 4.02 (w);
b) (+)- or (−)-erythro-mefloquine hydrochloride comprising particles having a size distribution of 30 to 150 μm, in a crystalline form which exhibits a characteristic X-ray powder diffraction pattern with peaks expressed in d-values (Å) of:
22.3 (vw), 11.2 (vs), 9.0 (w), 8.2 (vw), 7.4 (vw), 6.8 (vw), 6.5 (vw), 6.3 (vw), 6.1 (vw), 6.0 (vw), 5.94 (vw), 5.61 (m), 5.42 (w), 4.89 (vw), 4.74 (w), 4.54 (w), 4.12 (s), 4.02 (w), 3.81 (vvs), 3.74 (vs), 3.70 (vw), 3.64 (w), 3.55 (w), 3.47, (vw), 3.40 (vw), 3.34 (vw), 3.31 (vw), 3.26 (vs), 3.11 (vw), 3.04 (w), 2.97 (vw), 2.94 (vw), 2.81 (vw), 2.75 (m), 2.71 (w), 2.69 (w), 2.64 (w), 2.62 (w), 2.54 (vw), 2.46 (vw), 2.43 (vw), 2.40 (vw), 2.35 (vw), 2.30 (vw), 2.27 (vw), 2.24 (vw), 2.22 (vw), 2.17 (vs), 2.08 (vw), 2.06 (vw), 2.04 (wv), 1.94 (w), 1.91 (vw) and 1.88 (vw);
c) (+)- or (−)-erythro-mefloquine hydrochloride comprising particles having a size distribution of 1 to 10 μm, in a crystalline form which exhibits a characteristic X-ray powder diffraction pattern with peaks expressed in d-values (Å) of:
11.2 (m), 9.0 (w), 8.30 (vw), 7.4 (vw), 6.8 (vw), 6.3 (w), 6.1 (vw), 6.0 (vw), 5.95 (vw), 5.59 (w), 5.42 (w), 4.91 (vw), 4.74 (w), 4.55 (vw), 4.16 (w), 4.12 (s), 4.03 (w), 3.82 (ws), 3.75 (w), 3.71 (w), 3.64 (w), 3.55 (w), 3.47 (vw), 3.40 (vw), 3.33 (w), 3.26 (w), 3.11 (vw), 3.04 (vw), 2.94 (vw), 2.75 (w), 2.71 (vw), 2.69 (vw), 2.64 (w), 2.62 (vw), 2.54 (vw), 2.46 (vw), 2.43 (vw), 2.40 (vw), 2.35 (vw), 2.30 (vw), 2.26 (vw), 2.22 (vw), 2.17 (w), 2.08 (vw), 2.06 (vw), 1.99 (vw), 1.91 (vw) and 1.89 (vw);
d) (+)- or (−)-erythro-mefloquine hydrochloride in a crystalline form which exhibits characteristic Raman bands, expressed in wave numbers (cm⁻¹), of:
1030.2 (w) and 85.4 (vs);
e) (+) or (−)-erythro-mefloquine hydrochloride in a crystalline form which exhibits characteristic Raman bands, expressed in wave numbers (cm⁻¹), of:
2877 (m), 1601 (s), 1585 (s), 1363 (vs), 1028.2 (w), 320 (m) and 118 (vs);
f) (+)- or (−)-erythro-mefloquine hydrochloride which, as an acetone solvate, is in the form of a crystalline pseudo-polymorph which exhibits characteristic Raman bands, expressed in wave numbers (cm⁻¹) of:
1602 (s), 1585 (s), 1363 (vs), 322 (m) and 118 (vs);
g) (+)- or (−)-erythro-mefloquine hydrochloride which, as a tetrahydrofuran solvate, is in the form of a crystalline pseudo-polymorph which exhibits characteristic Raman bands, expressed in wave numbers (cm⁻¹), of:
1601 (s), 1585 (s), 1363 (vs), 323 (m) and 119 (vs);
h) (+)- or (−)-erythro-mefloquine hydrochloride which, as a methyl ethyl ketone solvate, exhibits characteristic Raman bands, expressed in wave numbers (cm⁻¹), of:
1600 (s), 1585 (s), 1363 (vs), 319 (m) and 118 (vs); and
i) (+)- or (−)-eythro-mefloquine hydrochloride, which is substantially in the form of thick columns, cuboids, cubes or cube-like particles.

46. The pharmaceutical composition, according to claim 45, comprising a (+)- or (−)-erythro-mefloquine hydrochloride having one or more of the following characteristics:
a) (+)- (−)-erythro-mefloquine hydrochloride in a crystalline forn which exhibits a characteristic X-ray powder diffraction pattern with peaks expressed in d-values (Å) of: 5.95 (s) and 4.02 (w);
b) (+)- or (−)-erythro-mefloquine hydrochloride comprising particles having a size distribution of 30 to 150 μm, in a crystaline form which exhibits a characteristic X-ray powder diffraction pattern with peaks expressed in d-values (Å) of:
22.3 (vw), 11.2 (vs), 9.0 (w), 8.2 (vw), 7.4 (vw), 6.8 (vw), 6.5 (vw), 6.3 (vw), 6.1 (vw), 6.0 (vw), 5.94 (vw), 5.61 (m), 5.42 (w), 4.89 (vw), 4.74 (w), 4.54 (w), 4.12 (s), 4.02 (w), 3.81 (vvs), 3.74 (vs), 3.70 (vw), 3.64 (w), 3.55 (w), 3.47, (vw), 3.40 (vw), 3.34 (vw), 3.31 (vw), 3.26 (vs), 3.11 (vw), 3.04 (w), 2.97 (vw), 2.94 (vw), 2.81 (vw), 2.75 (m), 2.71 (w), 2.69 (w), 2.64 (w), 2.62 (w), 2.54 (vw), 2.46 (vw), 2.43 (vw), 2.40 (vw), 2.35 (vw), 2.30 (vw), 2.27 (vw), 2.24 (vw), 2.22 (vw), 2.17 (vs), 2.08 (vw), 2.06 (vw), 2.04 (vw), 1.94 (w), 1.91 (vw) and 1.88 (vw);
c) (+)- or (−)-erythro-mefloquine hydrochloride comprising particles having a size distribution of 1 to 10 μm, in a crystalline form which exhibits a characteristic X-ray powder diffraction pattern with peaks expressed in d-values (Å) of:
11.2 (m), 9.0 (w), 8.30 (vw), 7.4 (vw), 6.8 (vw), 6.3 (w), 6.1 (vw), 6.0 (vw), 5.95 (vw), 5.59 (w), 5.42 (w), 4.91 (vw), 4.74 (w), 4.55 (vw), 4.16 (w), 4.12 (s), 4.03 (w), 3.82 (vvs), 3.75 (w), 3.71 ((w), 3.64 (w), 3.55 (w), 3.47 (vw), 3.40 (vw), 3.33 (w), 3.26 (w), 3.11 (vw), 3.04 (vw), 2.94 (vw), 2.75 (w), 2.71 (vw), 2.69 (vw), 2.64 (w), 2.62 (vw), 2.54 (vw), 2.46 (vw), 2.43 (vw), 2.40 (vw), 2.35 (vw), 2.30 (vw), 2.26 (vw), 2.22 (vw), 2.17 (w), 2.08 (vw), 2.06 (vw), 1.99 (vw), 1,91 (vw) and 1.89 (vw); and
d) (+)- or (−)-erythro-mefloquine hydrochloride in a crystalline form which exhibits characteristic Raman bands, expressed in wave numbers (cm⁻¹), of:
1030.2 (w) and 85.4 (vs).

47. A method for the treatment of malaria, a movement or neurodegenerative disorder, or an inflammatory or autoimmune disease wherein said method comprises administering, to a patient in need of such treatment, a (+)- or (−)-erythro-mefloquine hydrochloride having one or more of the following characteristics:
a) (+)- or (−)-erythro-mefloquine hydrochloride in a crystalline form which exhibits a characteristic X-ray powder diffraction pattern with peaks expressed in d-values (Å) of: 5.95 (s) and 4.02 (w);
b) (+)- or (−)-erythro-mefloquine hydrochloride comprising partcles having a size distribution of 30 to 150 μm in a crystalline form which exhibits a characteristic X-ray powder diffraction pattern with peaks expressed in d-values (Å) of:
22.3 (vw), 11.2 (vs), 9.0 (w), 8.2 (vw), 7.4 (vw), 6.8 (vw), 6.5 (vw), 6.3 (vw), 6.1 (vw), 6.0 (vw), 5.94 (vw), 5.61(m), 5.42 (w) 4.89 (vw), 4.74 (w), 4.54 (w), 4.12 (s), 4.02 (w), 3.81 (ws), 3.74 (vs), 3.70 (vw), 3.64 (w), 3.55 (w), 3.47, (vw), 3.40 (vw), 3.34 (vw), 3.31 (vw), 3.26 (vs), 3.11 (vw), 3.04 (w), 2.97 (vw), 2.94 (vw), 2.81 (vw), 2.75 (m), 2.71 (w), 2.69 (w), 2.64 (w), 2.62 (w), 2.54 (vw), 2.46 (vw), 2.43 (vw), 240 (vw), 2.35 (vw), 2.30 (v), 2.27 (vw), 2.24 (vw), 2.22 (vw), 2.17 (vs), 2.08 (vw), 2.06 (vw), 2.04 (vw), 1.94 (w), 1.91 (vw) and 1.88 (vw);
c) (+)- or (−)-erythro-mefloquine hydrochloride comprising particles having a size distribution of 1 to 10 μm, in a crstalline form which exhibits a characteristic X-ray powder diffraction pattern with peaks expressed in d-values (Å) of:
11.2 (m), 9.0 (w), 8.30 (vw), 7.4 (vw), 6.8 (vw), 6.3 (w), 6.1 (vw), 6.0 (vw), 5.95 (vw), 5.59 (w), 5.42 (w), 4.91 (vw), 4.74 (w), 4.55 (vw), 4.16 (w), 4.12 (s), 4.03 (w), 3.82 (ws), 3.75 (w), 3.71 (w), 3.64 (w), 3.55 (w), 3.47 (vw), 3.40 (vw), 3.33 (w), 3.26 (w), 3.11 (vw), 3.04 (vw), 2.94 (vw), 2.75 (w), 2.71 (vw), 2.69 (vw), 2.64 (w), 2.62 (vw), 2.54 (vw), 2.46 (vw), 2.43 (vw), 2.40 (vw), 2.35 (vw), 2.30 (vw), 2.26 (vw), 2.22 (vw), 2.17 (w), 2.08 (vw), 2.06 (vw), 1.99 (vw), 1.91 (vw) and 1.89 (vw);
d) (+)- or (−)-erythro-mefloquine hydrochloride in a crystalline form which exhibits characteristic Raman bands, expressed in wave numbers (cm⁻¹), of:
1030.2 (w) and 85.4 (vs);
e) (+)- or (−)-erythro-mefloquine hydrochloride in a crystalline form which exhibits characteristic Raman bands, expressed in wave numbers (cm⁻¹), of:
2877 (m), 1601 (s), 1585 (s), 1363 (vs), 1028.2 (w), 320 (m) and 118 (vs);
f) (+)- or (−)-erythro-mefloquine hydrochloride which, as an acetone solvate, is in the form of a crystalline pseudo-polymorph which exhibits characteristic Raman bands, expressed in wave numbers (cm⁻¹), of:
1602 (s), 1585 (s), 1363 (vs), 322 (m) and 118 (vs);
g) (+)- or (−)-erythro-mefloquine hydrochloride which, as a tetrrhydrofuran solvate, is in the form of a crystalline pseudo-polymorph which exhibits characteristic Raman bands, expressed in wave numbers (cm⁻¹), of:
1601 (s), 1585 (s), 1363 (vs), 323 (m) and 119 (vs);
h) (+)- or (−)-erythro-mefloquine hydrochloride which, as a methyl ethyl ketone solvate, exhibits characteristic Raman bands, expressed in wave numbers (cm⁻¹), of:
1600 (s), 1585 (s), 1363 (vs), 319 (m) and 118 (vs); and
i) (+)- or (−)-erythro-mefloquine hydrochloride, which is substantially in the form of thick columns, cuboids, cubes or cube-like particles.

48. The method, according to claim 47, which comprises administering a (+)- or (−)-erythro-mefloquine hydrochloride having one or more of the following characteristics:
a) (+)- or (−)-erythro-mefloquine hydrochloride in a crystalline form which exhibits a characteristic X-ray powder diffraction pattern with peaks expressed in d-values (Å) of: 5.95 (s) and 4.02 (w);
b) (+)- or (−)-erythro-mefloquine hydrochloride comprising particles having a size distribution of 30 to 150 μm, in a crystalline form which exhibits a X-ray powder diffraction pattern with peaks expressed in d-values (Å) of:
22.3 (vw), 11.2 (vs), 9.0 (w), 8.2 (vw), 7.4 (vw), 6.8 (vw), 6.5 (vw), 6.3 (vw), 6.1 (vw), 6.0 (vw), 5.94 (vw), 5.61 (m), 5.42 (w), 4.89 (vw), 4.74 (w), 4.54 (w), 4.12 (s), 4.02 (w), 3.81 (ws), 3.74 (vs), 3.70 (vw), 3.64 (w), 3.55 (w), 3.47, (vw), 3.40 (vw), 3.34 (vw), 3.31 (vw), 3.26 (vs), 3.11 (vw), 3.04 (w), 2.97 (vw), 2.94 (vw), 2.81 (vw), 2.75 (m), 2.71 (w), 2.69 (w), 2.64 (w), 2.62 (w), 2.54 (vw), 2.46 (vw), 2.43 (vw), 2.40 (vw), 2.35 (vw), 2.30 (vw), 2.27 (vw), 2.24 (vw), 2.22 (vw), 2.17 (vs), 2.08 (vw), 2.06 (vw), 2.04 (vw), 1.94 (w), 1.91 (vw) and 1.88 (vw);
c) (+)- or (−)-erythro-mefloquine hydrochloride comprising particles having a size distribution of 1 to 10 μm, in a crystalline form which exhibits a characteristic X-ray powder diffaction pattern with peaks expressed in d-values (Å) of:
11.2 (m), 9.0 (w), 8.30 (vw), 7.4 (vw), 6.8 (vw), 6.3 (w), 6.1 (vw), 6.0 (vw), 5.95 (vw), 5.59 (w), 5.42 (w), 4.91 (vw), 4.74 (w), 4.55 (vw), 4.16 (w), 4.12 (s), 4.03 (w), 3.82 (ws), 3.75 (w), 3.71 (w), 3.64 (w), 3.55 (w), 3.47 (vw), 3.40 (vw), 3.33 (w), 3.26 (w), 3.11 (vw), 3.04 (vw), 2.94 (vw), 2.75 (w), 2.71 (vw), 2.69 (vw), 2.64 (w), 2.62 (vw), 2.54 (vw), 2.46 (vw), 2.43 (vw), 2.40 (vw), 2.35 (vw), 2.30 (vw), 2.26 (vw), 2.22 (vw), 2.17 (w), 2.08 (vw), 2.06 (vw), 1.99 (vW), 1.91 (vw) and 1.89 (vw); and
d) (+)- or (−)-erythro-mefloquine hydrochloride in a crystalline form which exhibits characteristic Raman bands, expressed in wave numbers (cm⁻¹), of:
1030.2 (w) and 85.4 (vs).
